(19) United States
(12) Patent Application Publication
(10) Pub. No.: US 2023/0078454 A1
(43) Pub. Date: Mar. 16, 2023

(54) USING MACHINE LEARNING TO OPTIMIZE ASSAYS FOR SINGLE CELL TARGETED SEQUENCING

(71) Applicant: Mission Bio, Inc., South San Francisco, CA (US)

(72) Inventors: Dongmyunghee Kim, South San Francisco, CA (US); Manimozhi Manivannan, South San Francisco, CA (US); Saurabh Gulati, South San Francisco, CA (US); Shu Wang, South San Francisco, CA (US)

(21) Appl. No.: 17/801,097

(22) PCT Filed: Feb. 21, 2021
(86) PCT No.: PCT/US2021/018944

§ 371 Date: Aug. 19, 2022

Related U.S. Application Data
(63) Continuation of application No. PCT/US2020/043154, filed on Jul. 22, 2020.
(60) Provisional application No. 62/979,840, filed on Feb. 21, 2020.
(60) Provisional application No. 62/877,263, filed on Jul. 22, 2019.

(51) Int. Cl. G16B 35/10; G16B 35/20; G16B 40/20; C12Q 1/6886

(52) U.S. Cl. CPC G16B 35/10; G16B 35/20; G16B 40/20; C12Q 1/6886

(57) ABSTRACT

Disclosed herein is an amplicon design workflow for improving the design of amplicons such that panels including newly designed amplicons can achieve improved performance (e.g., improved panel uniformity). The amplicon design workflow involves performing a feature selection process to identify key amplicon attributes that likely lead to improved amplicon performance. Therefore, improved amplicons can be designed based on these key attributes. A sequencing panel, such as a DNA sequencing panel or RNA sequencing panel can be constructed using these improved amplicons and further validated. Thus, such panels including improved amplicons can be deployed for analyzing single cells e.g., through a single cell workflow analysis, for characterizing the cells for nucleic acid events, such as the presence or absence of RNA fusion transcripts.

### CROSS-REFERENCE TO RELATED APPLICATIONS

This application claims the benefit of and priority to the Provisional Application No. 62/979,840 filed Feb. 21, 2020, and PCT/US2020/043154 filed Jul. 22, 2020, each of which is hereby incorporated by reference in its entirety for all purposes.

### BACKGROUND

High throughput single-cell sequencing allows for interrogation of individual cells at genomic DNA and/or RNA levels. However, a standing challenge with sequencing at single-cell level is the non-uniform amplification which results in inadequate coverage of targets of interest. Thus, there is a need for automated workflows for designing improved sequencing panels such that the improved sequencing panels can achieve better performance

### SUMMARY

Disclosed herein is an amplicon design workflow for optimizing amplicon design to improve performance of sequencing panels. In various embodiments, the amplicon design workflow involves implementing a machine learning technique for identifying key amplicon attributes that likely lead to improved amplicon performance (e.g., improved panel uniformity). Thus, improved amplicons can be designed using these key attributes to be included in a sequencing panel, such as a DNA sequencing panel or RNA sequencing panel. In various embodiments, the panel including the improved amplicons can be validated. Thus, after validation, the panel including the improved amplicons can be deployed for analyzing single cells e.g., through a single cell workflow analysis. Analyzing single cells can include characterizing the cells for nucleic acid events, such as the presence or absence of RNA fusion transcripts.

Disclosed herein is a method for designing a panel of RNA fusion amplicons, the method comprising: providing a plurality of RNA fusion amplicons having a plurality of initial attributes, the RNA fusion amplicons representing one or more RNA fusions; sequencing the plurality of RNA fusion amplicons with a targeted RNA panel; selecting a subset of the plurality of RNA fusion amplicons according to performance of the subset of RNA fusion amplicons; performing a feature selection among the subset of RNA fusion amplicons to select key attributes from the plurality of initial attributes, and designing a plurality of improved RNA fusion amplicons comprising candidate attributes that are selected based on the key attributes of the subset of RNA fusion amplicons; and validating the plurality of improved RNA fusion amplicons.

In various embodiments, performing a feature selection among the subset of RNA fusion amplicons to select key attributes from the plurality of initial attributes further comprises applying a ranking model. In various embodiments, the ranking model implements a Recursive Feature Elimination (RFE) technique. In various embodiments, performing a feature selection among the subset of RNA fusion amplicons to select key attributes from the plurality of initial attributes further comprises applying a second model. In various embodiments, the second model comprises a weighted model. In various embodiments, the selected key attributes represent attributes that are selected by both the ranking model and the second model. In various embodiments, performing the feature selection further comprises: selecting key attributes representing independent attributes from highest importance attributes.

In various embodiments, comprising calculating a plurality of statistical parameters from the key attributes. In various embodiments, designing the plurality of improved RNA fusion amplicons comprising attributes that are selected based on the key attributes comprises designing the plurality of improved RNA fusion amplicons to include one or more of the plurality of statistical parameters calculated from the key attributes. In various embodiments, validating the plurality of improved RNA fusion amplicons comprises sequencing the plurality of improved RNA fusion amplicons and determining a performance of the improved RNA fusion amplicons. In various embodiments, validating the plurality of improved RNA fusion amplicons comprises applying a predictive model to the plurality of improved RNA fusion amplicons, the predictive model trained to predict a performance of RNA fusion amplicons.

In various embodiments, the performance is a measure of panel uniformity. In various embodiments, the performance is a sensitivity or specificity of detection of a presence or absence of a RNA fusion using the plurality of improved RNA fusion amplicons. In various embodiments, providing the plurality of RNA fusion amplicons having a plurality of initial attributes comprises constructing at least one fusion sequence. In various embodiments, constructing the at least one fusion sequence comprises: obtaining a sequence of a first gene and a sequence of a second gene; identifying a fusion breakpoint in the sequence for the first gene and a fusion breakpoint in the sequence for the second gene; concatenating the sequence of the first gene at the fusion breakpoint for the first gene with the sequence of the second gene at the fusion breakpoint for the second gene; stitching together exon sequences of the first gene and the exon sequences of the second gene that flank the concatenated sequences at the fusion breakpoints.

Additionally disclosed herein is a method for designing a panel of amplicons, the method comprising: providing a plurality of amplicons having a plurality of initial attributes; sequencing the plurality of amplicons with a single cell panel; selecting a subset of the plurality of amplicons according to performance of the subset of amplicons; performing a feature selection among the subset of amplicons to select key attributes from the plurality of initial attributes, and designing a plurality of improved amplicons wherein the improved amplicons comprise attributes designed based on the selected key attributes of the subset of amplicons; and validating the plurality of secondary amplicons. In various embodiments, performing a feature selection among the subset of amplicons to select key attributes from the plurality of initial attributes further comprises applying a ranking model. In various embodiments, the ranking model implements a Recursive Feature Elimination (RFE) technique. In various embodiments, performing a feature selection among the subset of amplicons to select key attributes from the plurality of initial attributes further comprises applying a second model. In various embodiments, the second model comprises a weighted model. In various embodiments, the selected key attributes represent attributes that are selected by both the ranking model and the second model.

In various embodiments, performing the feature selection further comprises: selecting key attributes representing independent attributes from highest importance attributes. In various embodiments, the method described above further comprises calculating a plurality of statistical parameters from the key attributes. In various embodiments, designing the plurality of improved amplicons comprising attributes that are selected based on the key attributes comprises designing the plurality of improved amplicons to include one or more of the plurality of statistical parameters calculated from the key attributes.

In various embodiments, validating the plurality of improved amplicons comprises sequencing the plurality of improved amplicons and determining a performance of the improved amplicons. In various embodiments, validating the plurality of improved amplicons comprises applying a predictive model to the plurality of improved amplicons, the predictive model trained to predict a performance of amplicons. In various embodiments, the performance is a measure of panel uniformity. In various embodiments, the performance is a sensitivity or specificity of detection of a presence or absence of a RNA fusion using the plurality of improved RNA fusion amplicons. In various embodiments, responsive to the validation determining that the plurality of improved amplicons fails to meet a pre-determined performance metric, re-analyzing the improved amplicons using an amplicon design workflow to generate further improved amplicons. In various embodiments, the single cell panel is a targeted RNA panel, a targeted DNA panel, a whole genome panel, or whole transcriptome panel. In various embodiments, the plurality of amplicons and the plurality of improved amplicons are DNA amplicons. In various embodiments, the plurality of amplicons and the plurality of improved amplicons are RNA fusion amplicons. In various embodiments, providing a plurality of amplicons having a plurality of initial attributes further comprises constructing at least one fusion sequence.

In various embodiments, constructing the at least one fusion sequence comprises: obtaining a sequence of a first gene and a sequence of a second gene; identifying a fusion breakpoint in the sequence for the first gene and a fusion breakpoint in the sequence for the second gene; concatenating the sequence of the first gene at the fusion breakpoint for the first gene with the sequence of the second gene at the fusion breakpoint for the second gene; stitching together exon sequences of the first gene and the exon sequences of the second gene that flank the concatenated sequences at the fusion breakpoints.

In various embodiments, the improved RNA fusion amplicons are designed according to a BCR-ABL RNA fusion. In various embodiments, the BCR-ABL RNA fusion is any one of a b3a2 RNA fusion, b2a2 RNA fusion, or e1a2 RNA fusion. In various embodiments, the BCR-ABL RNA fusion is a b3a2 RNA fusion, and wherein the improved RNA fusion amplicons achieve at least a 90% sensitivity. In various embodiments, the BCR-ABL RNA fusion is a b3a2 RNA fusion, and wherein the improved RNA fusion amplicons achieve at least a 90% specificity. In various embodiments, the BCR-ABL RNA fusion is a b2a2 RNA fusion, and wherein the improved RNA fusion amplicons achieve at least a 90% sensitivity. In various embodiments, the BCR-ABL RNA fusion is a b2a2 RNA fusion, and wherein the improved RNA fusion amplicons achieve at least a 90% specificity. In various embodiments, the BCR-ABL RNA fusion is a e1a2 RNA fusion, and wherein the improved RNA fusion amplicons achieve at least a 70% sensitivity. In various embodiments, the BCR-ABL RNA fusion is a e1a2 RNA fusion, and wherein the improved RNA fusion amplicons achieve at least a 90% specificity.

In various embodiments, the initial attributes, key attributes, or candidate attributes of amplicons comprise characteristics of primers that are designed to target the amplicons. In various embodiments, the initial attributes, key attributes, or candidate attributes are selected from a group consisting of a primer length, a percentage of GC content in a primer, a GC content at 3′ end of primer, a GC content at 5′ end of primer and a number of G or C bases within the last five bases of 3′ end of the primer.

Additionally disclosed herein is a non-transitory computer readable medium for designing a panel of RNA fusion amplicons, the non-transitory computer readable medium comprising instructions that, when executed by a processor, cause the processor to: provide a plurality of RNA fusion amplicons having a plurality of initial attributes, the RNA fusion amplicons representing one or more RNA fusions; sequence the plurality of RNA fusion amplicons with a targeted RNA panel; select a subset of the plurality of RNA fusion amplicons according to performance of the subset of RNA fusion amplicons; perform a feature selection among the subset of RNA fusion amplicons to select key attributes from the plurality of initial attributes, and design a plurality of improved RNA fusion amplicons comprising candidate attributes that are selected based on the key attributes of the subset of RNA fusion amplicons; and validate the plurality of improved RNA fusion amplicons.

In various embodiments, the instructions that, when executed by a processor, cause the processor to perform a feature selection among the subset of RNA fusion amplicons to select key attributes from the plurality of initial attributes further comprises instructions that, when executed by the processor, cause the processor to apply a ranking model. In various embodiments, the ranking model implements a Recursive Feature Elimination (RFE) technique. In various embodiments, the instructions that, when executed by a processor, cause the processor to perform a feature selection among the subset of RNA fusion amplicons to select key attributes from the plurality of initial attributes further comprises instructions that, when executed by the processor, cause the processor to apply a second model. In various embodiments, the second model comprises a weighted model. In various embodiments, the selected key attributes represent attributes that are selected by both the ranking model and the second model.

In various embodiments, the instructions that, when executed by a processor, cause the processor to perform the feature selection further comprises instructions that, when executed by the processor, cause the processor to: select key attributes representing independent attributes from highest importance attributes. In various embodiments, the instructions further comprise instructions that, when executed by the processor, cause the processor to calculate a plurality of statistical parameters from the key attributes. In various embodiments, the instructions that, when executed by a processor, cause the processor to design the plurality of improved RNA fusion amplicons comprising attributes that are selected based on the key attributes further comprises instructions that, when executed by the processor, cause the processor to design the plurality of improved RNA fusion amplicons to include one or more of the plurality of statistical parameters calculated from the key attributes.

In various embodiments, the instructions that, when executed by a processor, cause the processor to validate the plurality of improved RNA fusion amplicons further comprises instructions that, when executed by the processor, cause the processor to sequence the plurality of improved RNA fusion amplicons and determine a performance of the improved RNA fusion amplicons. In various embodiments, the instructions that, when executed by a processor, cause the processor to validate the plurality of improved RNA fusion amplicons further comprises instructions that, when executed by the processor, cause the processor to apply a predictive model to the plurality of improved RNA fusion amplicons, the predictive model trained to predict a performance of RNA fusion amplicons.

In various embodiments, the performance is a measure of panel uniformity. In various embodiments, the performance is a sensitivity or specificity of detection of a presence or absence of a RNA fusion using the plurality of improved RNA fusion amplicons. In various embodiments, the instructions that cause the processor to provide the plurality of RNA fusion amplicons having a plurality of initial attributes further comprises instructions that, when executed by the processor, cause the processor to construct at least one fusion sequence. In various embodiments, the instructions that, when executed by a processor, cause the processor to construct the at least one fusion sequence further comprises instructions that, when executed by the processor, cause the processor to: obtain a sequence of a first gene and a sequence of a second gene; identify a fusion breakpoint in the sequence for the first gene and a fusion breakpoint in the sequence for the second gene; concatenate the sequence of the first gene at the fusion breakpoint for the first gene with the sequence of the second gene at the fusion breakpoint for the second gene; and stitch together exon sequences of the first gene and the exon sequences of the second gene that flank the concatenated sequences at the fusion breakpoints.

Additionally disclosed herein is a non-transitory computer readable medium for designing a panel of amplicons comprising instructions that, when executed by a processor, cause the processor to: provide a plurality of amplicons having a plurality of initial attributes; sequence the plurality of amplicons with a single cell panel; select a subset of the plurality of amplicons according to performance of the subset of amplicons; perform a feature selection among the subset of amplicons to select key attributes from the plurality of initial attributes, and design a plurality of improved amplicons wherein the improved amplicons comprise attributes designed based on the selected key attributes of the subset of amplicons; and validate the plurality of secondary amplicons.

In various embodiments, the instructions that cause the processor to perform a feature selection among the subset of amplicons to select key attributes from the plurality of initial attributes further comprises instructions that, when executed by the processor, cause the processor to apply a ranking model. In various embodiments, the ranking model implements a Recursive Feature Elimination (RFE) technique. In various embodiments, the instructions that cause the processor to perform a feature selection among the subset of amplicons to select key attributes from the plurality of initial attributes further comprises instructions that, when executed by the processor, cause the processor to apply a second model. In various embodiments, the second model comprises a weighted model. In various embodiments, the selected key attributes represent attributes that are selected by both the ranking model and the second model.

In various embodiments, the instructions that cause the processor to perform the feature selection further comprises instructions that, when executed by the processor, cause the processor to: select key attributes representing independent attributes from highest importance attributes. In various embodiments, the instructions further comprise instructions that, when executed by a processor, cause the processor to calculate a plurality of statistical parameters from the key attributes. In various embodiments, the instructions that cause the processor to design the plurality of improved amplicons comprising attributes that are selected based on the key attributes further comprises instructions that, when executed by the processor, cause the processor to design the plurality of improved amplicons to include one or more of the plurality of statistical parameters calculated from the key attributes.

In various embodiments, the instructions that cause the processor to validate the plurality of improved amplicons further comprises instructions that, when executed by the processor, cause the processor to sequence the plurality of improved amplicons and determine a performance of the improved amplicons. In various embodiments, that cause the processor to validate the plurality of improved amplicons further comprises instructions that, when executed by the processor, cause the processor to apply a predictive model to the plurality of improved amplicons, the predictive model trained to predict a performance of amplicons.

In various embodiments, the performance is a measure of panel uniformity. In various embodiments, the performance is a sensitivity or specificity of detection of a presence or absence of a RNA fusion using the plurality of improved RNA fusion amplicons. In various embodiments, responsive to the validation determining that the plurality of improved amplicons fails to meet a pre-determined performance metric, the instructions, when executed by the processor, cause the processor to re-analyze the improved amplicons using an amplicon design workflow to generate further improved amplicons. In various embodiments, the single cell panel is a targeted RNA panel, a targeted DNA panel, a whole genome panel, or whole transcriptome panel. In various embodiments, the plurality of amplicons and the plurality of improved amplicons are DNA amplicons. In various embodiments, the plurality of amplicons and the plurality of improved amplicons are RNA fusion amplicons.

In various embodiments, the instructions that cause the processor to provide a plurality of amplicons having a plurality of initial attributes further comprises instructions that when executed by the processor, cause the processor to construct at least one fusion sequence. In various embodiments, the instructions that cause the processor to construct the at least one fusion sequence further comprises instructions that when executed by the processor, cause the processor to: obtain a sequence of a first gene and a sequence of a second gene; identify a fusion breakpoint in the sequence for the first gene and a fusion breakpoint in the sequence for the second gene; concatenate the sequence of the first gene at the fusion breakpoint for the first gene with the sequence of the second gene at the fusion breakpoint for the second gene; stitch together exon sequences of the first gene and the exon sequences of the second gene that flank the concatenated sequences at the fusion breakpoints.

In various embodiments, the improved RNA fusion amplicons are designed according to a BCR-ABL RNA fusion. In various embodiments, the BCR-ABL RNA fusion is any one of a b3a2 RNA fusion, b2a2 RNA fusion, or e1a2 RNA fusion. In various embodiments, the BCR-ABL RNA fusion is a b3a2 RNA fusion, and wherein the improved RNA fusion amplicons achieve at least a 90% sensitivity. In various embodiments, the BCR-ABL RNA fusion is a b3a2 RNA fusion, and wherein the improved RNA fusion amplicons achieve at least a 90% specificity. In various embodiments, the BCR-ABL RNA fusion is a b2a2 RNA fusion, and wherein the improved RNA fusion amplicons achieve at least a 90% sensitivity. In various embodiments, the BCR-ABL RNA fusion is a b2a2 RNA fusion, and wherein the improved RNA fusion amplicons achieve at least a 90% specificity. In various embodiments, the BCR-ABL RNA fusion is a e1a2 RNA fusion, and wherein the improved RNA fusion amplicons achieve at least a 70% sensitivity. In various embodiments, the BCR-ABL RNA fusion is a e1a2 RNA fusion, and wherein the improved RNA fusion amplicons achieve at least a 90% specificity. In various embodiments, the initial attributes, key attributes, or candidate attributes of amplicons comprise characteristics of primers that are designed to target the amplicons. In various embodiments, the initial attributes, key attributes, or candidate attributes are selected from a group consisting of a primer length, a percentage of GC content in a primer, a GC content at 3′ end of primer, a GC content at 5′ end of primer and a number of G or C bases within the last five bases of 3′ end of the primer.

### BRIEF DESCRIPTION OF THE DRAWINGS

These and other features, aspects, and advantages of the present invention will become better understood with regard to the following description and accompanying drawings. It is noted that wherever practicable similar or like reference numbers may be used in the figures and may indicate similar or like functionality. For example, a letter after a reference numeral, such as “third party entity 130A,” indicates that the text refers specifically to the element having that particular reference numeral. A reference numeral in the text without a following letter, such as “third party entity 130,” refers to any or all of the elements in the figures bearing that reference numeral (e.g. “third party entity 130” in the text refers to reference numerals “third party entity 130A” and/or “third party entity 130B” in the figures).

FIG. 1 depicts a system environment including a panel design system, in accordance with an embodiment.

FIG. 2 depicts an example flow diagram for designing amplicons, in accordance with an embodiment.

FIG. 3A depicts an example flow diagram for constructing a fusion sequence, in accordance with an embodiment.

FIG. 3B is an example schematic for constructing a fusion sequence, in accordance with an embodiment.

FIG. 3C depicts an example flow diagram for performing a feature selection process to identify key attributes of amplicons, in accordance with an embodiment.

FIG. 4 depicts an example computing device for implementing system and methods described in reference to FIGS. 1-3A/3B.

FIG. 5 depicts example box plots showing different categories (e.g., low, average, high) of amplicons based on values for four different amplicon features.

FIG. 6 depicts example correlation between different amplicon features.

FIG. 7A shows an example process including feature selection of key attributes and in silico validation of amplicons designed based on the key attributes.

FIG. 7B depicts performance data (e.g., accuracy and F1 score) of the prediction model that was trained on differing panels (e.g., small versus large panels). Two ML classification models (KNC and SVC) with K-fold cross validation were trained with 10000 splits of 70/30 for training/testing dataset split, while all splits keep the same ratio of classes in both training and testing datasets. Average accuracy ranges from 0.80-0.88 for large dataset to 0.90-0.98 for small panels.

FIG. 7C depicts example performance data (e.g., panel uniformity) of the prediction model across differently sized panels. Specifically, implementing the amplicon designer workflow significantly improved amplicon performance and uniformity in targeted assay design across different panel size and genomic contents (human and mouse genomes). Three (3) newly designed panels were sequenced. Multiple runs were conducted for each panel.

FIG. 8A depicts a heat map for a DNA panel using RNA fusion amplicons that were designed using the amplicon design workflow.

FIG. 8B depicts performance (e.g., sensitivity and specificity) metrics for detecting three different RNA fusions using the amplicon design workflow.

### DETAILED DESCRIPTION

### Definitions

Various aspects of the invention will now be described with reference to the following section which will be understood to be provided by way of illustration only and not to constitute a limitation on the scope of the invention.

“Complementarity” refers to the ability of a nucleic acid to form hydrogen bond(s) or hybridize with another nucleic acid sequence by either traditional Watson-Crick or other non-traditional types. As used herein “hybridization,” refers to the binding, duplexing, or hybridizing of a molecule only to a particular nucleotide sequence under low, medium, or highly stringent conditions, including when that sequence is present in a complex mixture (e.g., total cellular) DNA or RNA. See e.g. Ausubel, et al., Current Protocols In Molecular Biology, John Wiley & Sons, New York, N.Y., 1993. If a nucleotide at a certain position of a polynucleotide is capable of forming a Watson-Crick pairing with a nucleotide at the same position in an anti-parallel DNA or RNA strand, then the polynucleotide and the DNA or RNA molecule are complementary to each other at that position. The polynucleotide and the DNA or RNA molecule are “substantially complementary” to each other when a sufficient number of corresponding positions in each molecule are occupied by nucleotides that can hybridize or anneal with each other in order to affect the desired process. A complementary sequence is a sequence capable of annealing under stringent conditions to provide a 3′-terminal serving as the origin of synthesis of complementary chain.

The terms “amplify”, “amplifying”, “amplification reaction” and their variants, refer generally to any action or process whereby at least a portion of a nucleic acid molecule (referred to as a template nucleic acid molecule) is replicated or copied into at least one additional nucleic acid molecule. The additional nucleic acid molecule optionally includes the sequence that is substantially identical or substantially complementary to at least some portion of the template nucleic acid molecule. The template nucleic acid molecule can be single-stranded or double-stranded and the additional nucleic acid molecule can independently be single-stranded or double-stranded. In some embodiments, amplification includes a template-dependent in vitro enzyme-catalyzed reaction for the production of at least one copy of at least some portion of the nucleic acid molecule or the production of at least one copy of a nucleic acid sequence that is complementary to at least some portion of the nucleic acid molecule Amplification optionally includes linear or exponential replication of a nucleic acid molecule. In some embodiments, such amplification is performed using isothermal conditions; in other embodiments, such amplification can include thermocycling. In some embodiments, the amplification is a multiplex amplification that includes the simultaneous amplification of a plurality of target sequences in a single amplification reaction. At least some of the target sequences can be situated, on the same nucleic acid molecule or on different target nucleic acid molecules included in the single amplification reaction. In some embodiments, “amplification” includes amplification of at least some portion of DNA- and RNA-based nucleic acids alone, or in combination. The amplification reaction can include single or double-stranded nucleic acid substrates and can further include any of the amplification processes known to one of ordinary skill in the art. In some embodiments, the amplification reaction includes polymerase chain reaction (PCR). Additionally, the terms “synthesis” and “amplification” of nucleic acid are used herein. The synthesis of nucleic acid in the present invention means the elongation or extension of nucleic acid from an oligonucleotide serving as the origin of synthesis. If not only this synthesis but also the formation of other nucleic acids and the elongation or extension reaction of this formed nucleic acid occur continuously, a series of these reactions is comprehensively called amplification. The polynucleic acid produced by the amplification technology employed is generically referred to as an “amplicon” or “amplification product.”

The terms “nucleic acid,” “polynucleotides,” and “oligonucleotides” refers to biopolymers of nucleotides and, unless the context indicates otherwise, includes modified and unmodified nucleotides, and both DNA and RNA, and modified nucleic acid backbones. For example, in certain embodiments, the nucleic acid is a peptide nucleic acid (PNA) or a locked nucleic acid (LNA). Typically, the methods as described herein are performed using DNA as the nucleic acid template for amplification. However, nucleic acid whose nucleotide is replaced by an artificial derivative or modified nucleic acid from natural DNA or RNA is also included in the nucleic acid of the present invention insofar as it functions as a template for synthesis of the complementary chain. The nucleic acid of the present invention is generally contained in a biological sample. The biological sample includes animal, plant or microbial tissues, cells, cultures and excretions, or extracts therefrom. In certain aspects, the biological sample includes intracellular parasitic genomic DNA or RNA such as virus or mycoplasma. The nucleic acid may be derived from nucleic acid contained in said biological sample. For example, genomic DNA, or cDNA synthesized from mRNA, or nucleic acid amplified on the basis of nucleic acid derived from the biological sample, are preferably used in the described methods. Unless denoted otherwise, whenever a oligonucleotide sequence is represented, it will be understood that the nucleotides are in 5′ to 3′ order from left to right and that “A” denotes deoxyadenosine, “C” denotes deoxycytidine, “G” denotes deoxyguanosine, “T” denotes thymidine, and “U’ denotes deoxyuridine. Oligonucleotides are said to have “5′ ends” and “3′ ends” because mononucleotides are typically reacted to form oligonucleotides via attachment of the 5′ phosphate or equivalent group of one nucleotide to the 3′ hydroxyl or equivalent group of its neighboring nucleotide, optionally via a phosphodiester or other suitable linkage.

A template nucleic acid is a nucleic acid serving as a template for synthesizing a complementary chain in a nucleic acid amplification technique. A complementary chain having a nucleotide sequence complementary to the template has a meaning as a chain corresponding to the template, but the relationship between the two is merely relative. That is, according to the methods described herein a chain synthesized as the complementary chain can function again as a template. That is, the complementary chain can become a template. In certain embodiments, the template is derived from a biological sample, e.g., plant, animal, virus, micro-organism, bacteria, fungus, etc. In certain embodiments, the animal is a mammal, e.g., a human patient. A template nucleic acid typically comprises one or more target nucleic acid. A target nucleic acid in exemplary embodiments may comprise any single or double-stranded nucleic acid sequence that can be amplified or synthesized according to the disclosure, including any nucleic acid sequence suspected or expected to be present in a sample.

Embodiments disclosed herein may select target nucleic acid sequences for genes corresponding to oncogenesis, such as oncogenes, proto-oncogenes, and tumor suppressor genes. In some embodiments the analysis includes the characterization of mutations, copy number variations, and other genetic alterations associated with oncogenesis. Any known proto-oncogene, oncogene, tumor suppressor gene or gene sequence associated with oncogenesis may be a target nucleic acid that is studied and characterized alone or as part of a panel of target nucleic acid sequences (e.g., target nucleic acid sequences in amplicons). For examples, see Lodish H, Berk A, Zipursky S L, et al. Molecular Cell Biology. 4th edition. New York: W. H. Freeman; 2000. Section 24.2, Proto-Oncogenes and Tumor-Suppressor Genes. Available from: https://www.ncbi.nlm.nih.gov/books/NBK21662/, incorporated by reference herein.

As used herein, the term “panel” refers to a group of amplicons that target a specific genome of interest or target a specific loci of interest on a genome.

The phrase “nucleic acid events” refers to one or more of polymorphisms, single nucleotide polymorphisms (SNPs), single nucleotide variants (SNVs)), insertions, deletions, knock-ins, knock-outs, copy number variations (CNVs), duplications, translocations, and loss of heterozygosity, or fusions. Nucleic acid events can refer to events in either DNA, such as genomic DNA, or RNA transcripts.

The phrase “amplicon attributes” and “amplicon features” are used interchangeably herein. In various embodiments, amplicon attributes refer to characteristics of primers that target the amplicon (e.g., primers that prime the amplicon and participate in nucleic acid amplification of the amplicon). In various embodiments, amplicon attributes refer to characteristics of the amplicon, including but not limited to the characteristics of the insert, which is the region of interest amplified by primers. In various embodiments, amplicon attributes include both characteristics of amplicons and characteristics of primers that target the amplicon.

The term “performance” used in the context of amplicon performance or panel performance refers to any of extent of coverage, panel uniformity, or normalized read value for an amplicon. Performance metrics can further include detection of a cell with a nucleic acid event, such as a RNA fusion. For example, performance metrics can include sensitivity and/or specificity of detecting cells with nucleic acid events.

### Overall System Environment

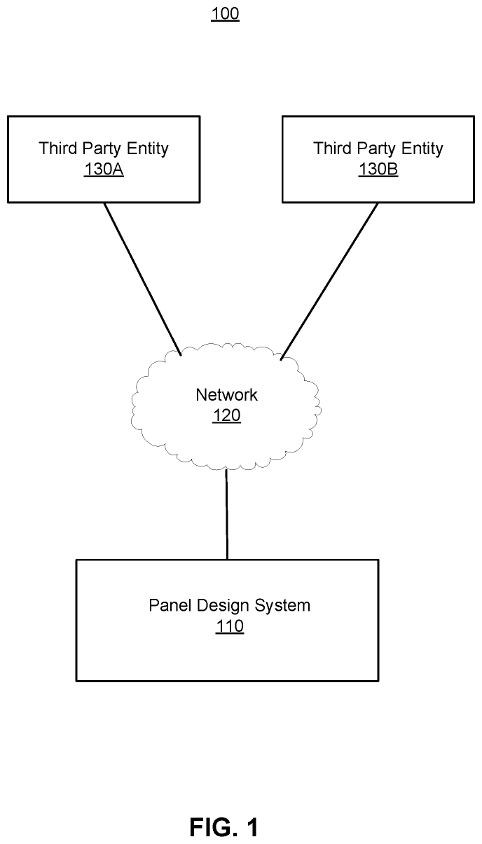

FIG. 1 depicts a system environment 100 including a panel design system 110, in accordance with an embodiment. The system environment 100 shown in FIG. 1 includes the panel design system 110 and one or more third party entities 130A and 130B in communication with one another through a network 120. In some embodiments, additional or fewer third party entities 130 in communication with the panel design system 110 can be included. The third party entities 130 communicate with the panel design system 110 for purposes associated with developing sequencing panels with designed amplicons. As one example, the panel design system 110 can develop custom sequencing panels with designed amplicons for individual third party entities 130. Therefore, a third party entity can implement the sequencing panel with the designed amplicons to perform analysis of single cells.

### Panel Design System

Generally, the panel design system 110 implements an amplicon design workflow to design amplicons for sequencing panels Implementing sequencing panels including the designed amplicons achieves improved metrics such as improved panel uniformity and/or increased detection of nucleic acid events (e.g., mutations present in genomic DNA or RNA transcripts, DNA or RNA fusions or translocations). Therefore, sequencing panels including the designed amplicons can be used to analyze individual cells (e.g., through a single-cell analysis involving DNA and/or RNA) to detect nucleic acid events.

In various embodiments, the amplicon design workflow performed by the panel design system 110 involves a feature selection process that identifies key attributes of amplicons that result in high-performing amplicons. In various embodiments, the amplicons are DNA amplicons and therefore, the feature selection process involves identifying key attributes of DNA amplicons that lead to high performance (e.g., high panel uniformity and/or detection of nucleic acid events in genomic DNA). In various embodiments, the amplicons are RNA amplicons and therefore, the feature selection process involves identifying key attributes of RNA amplicons that lead to high performance (e.g., high panel uniformity and/or detection of nucleic acid events in RNA transcripts). As used herein, “RNA amplicons” refers to amplicons derived from RNA transcripts. For example, RNA amplicons can be cDNA amplicons. Here, a RNA amplicon can be reverse transcribed to generate a cDNA nucleic acid and the cDNA nucleic acid can undergo nucleic acid amplification to generate cDNA amplicons.

In some embodiments, RNA amplicons are RNA fusion amplicons that are designed to detect the presence of RNA fusions (e.g., presence of RNA fusions in RNA fusion transcripts). In various embodiments, the amplicon design workflow includes designing improved amplicons based on identified key attributes of amplicons that lead to high-performing amplicons. For example, the newly designed amplicons incorporate aspects of the key attributes of high-performing amplicons and therefore, the newly designed amplicons are likely to be similarly high performing when subsequently implemented in a sequencing panel. In various embodiments, the amplicon design workflow involves validating the newly designed amplicons validate their performance For example, the amplicons can be generated and sequenced using a sequencing panel to determine metrics such as panel uniformity and/or detection of nucleic acid events (e.g., mutations in genomic DNA and/or RNA fusion events in RNA transcripts). Validated amplicons can be included in a sequencing panel. In various embodiments, a sequencing panel can be a custom sequencing panel designed for a party (e.g., such as a third party entity 130). In various embodiments, the sequencing panel can be implemented by the panel design system 110 for subsequent cellular analysis, such as single-cell analysis.

### Third Party Entity

In various embodiments, a third party entity 130 (e.g., third party entity 130A or third party entity 130B) represents a partner entity of the panel design system 110 that operates either upstream or downstream of the panel design system 110. As one example, the third party entity 130 operates upstream of the panel design system 110 and provides information to the panel design system 110 to enable the implementation of the amplicon design workflow. In this scenario, the panel design system 110 receives data from the third party entity 130. In various embodiments, the received data includes amplicons with initial attributes. Examples of amplicons with initial attributes is described in further detail below (e.g., Tables 1). For example, the data including amplicons with initial attributes can correspond to a custom sequencing panel. In various embodiments, the received data includes sequencing data pertaining to amplicons with initial attributes. In various embodiments, the received data includes metrics describing performance of amplicons with initial attributes. Thus, the panel design system 110 can use the data received from the third party entity 130 to identify key attributes of the amplicons, and design improved amplicons based on the identified key attributes. The new panels including the improved amplicons exhibit improved performance in comparison to an initial panel including amplicons with initial attributes.

In various embodiments, the third party entity 130 operates downstream of the panel design system 110 and receives information from the panel design system 110 pertaining to new panels including improved amplicons. In this scenario, the panel design system 110 may implement the amplicon design workflow to generate the new panels including improved amplicons. In various embodiments, the panel design system 110 provides the design of the improved amplicons to the third party entity 130. Therefore, the third party entity 130 can perform cellular analysis using the new panels including the improved amplicons. In various embodiments, the panel design system 110 can implement the new panels with the improved amplicons to analyze cells, and can provide the results of the cellular analysis to the third party entity 130. Here, the results of the cellular analysis generated using the new panels with the improved amplicons represents an improvement (e.g., improved panel uniformity, improved detection such as sensitivity or specificity) in comparison to a cellular analysis generated using panels including amplicons that were not generated using the amplicon design workflow (e.g., panels including amplicons with the initial attributes).

### Network

This disclosure contemplates any suitable network 120 that enables connection between the panel design system 110 and third party entities 130. The network 120 may comprise any combination of local area and/or wide area networks, using both wired and/or wireless communication systems. In one embodiment, the network 120 uses standard communications technologies and/or protocols. For example, the network 120 includes communication links using technologies such as Ethernet, 802.11, worldwide interoperability for microwave access (WiMAX), 3G, 4G, code division multiple access (CDMA), digital subscriber line (DSL), etc. Examples of networking protocols used for communicating via the network 120 include multiprotocol label switching (MPLS), transmission control protocol/Internet protocol (TCP/IP), hypertext transport protocol (HTTP), simple mail transfer protocol (SMTP), and file transfer protocol (FTP). Data exchanged over the network 120 may be represented using any suitable format, such as hypertext markup language (HTML) or extensible markup language (XML). In some embodiments, all or some of the communication links of the network 120 may be encrypted using any suitable technique or techniques.

### Methods for Amplicon Design Workflow

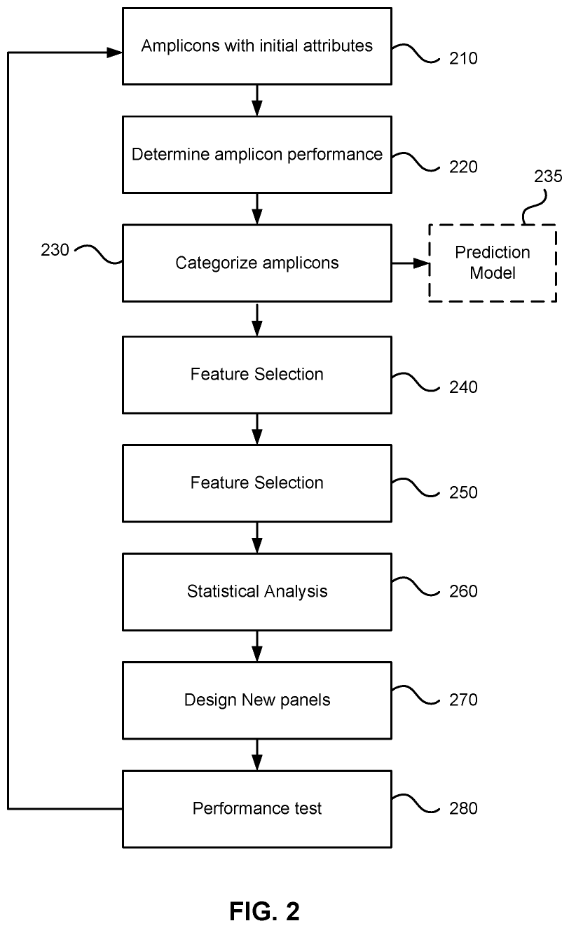

FIG. 2 depicts an example flow diagram for designing amplicons, in accordance with an embodiment. Generally, FIG. 2 depicts the amplicon design workflow that involves identifying key attributes of amplicons through a feature selection process, and designing improved amplicons based on the identified key attributes. Thus, a panel including the improved amplicons achieves improved performance (e.g., improved panel uniformity, improved sensitivity, and/or improved specificity when detecting nucleic acid events).

In various embodiments, the amplicon design workflow includes steps 210, 220, 230, 235, 240, 250, 260, 270, and 280. In various embodiments, step 235 involving the prediction model is optional and need not be implemented. In various embodiments, the amplicon design workflow includes a subset of steps 210, 220, 230, 235, 240, 250, 260, 270, and 280. In some embodiments, the amplicon design workflow need not include steps 210 and 220. For example, steps 210 and 220 can be performed by a third party (e.g., third party entity 130 described in FIG. 1) such that the amplicon design workflow begins at step 230 by selecting a subset of the amplicons based on amplicon performance provided by the third party system. In various embodiments, the amplicon design workflow includes only one feature selection step (e.g., only one of step 240 or 250) as opposed to the two feature selection steps shown in FIG. 2.

At step 210, amplicons with initial attributes are designed. In various embodiments, multiple panels with various sizes can be designed with amplicons spanning a wide range of attributes. In various embodiments, here at step 210, the attributes of the amplicons, hereafter referred to as initial attributes, were not determined using the amplicon design workflow described herein.

In various embodiments, step 210 involves designing amplicons with initial attributes for a DNA sequencing panel. In various embodiments, step 210 involves designing amplicons with initial attributes for a RNA sequencing panel. In various embodiments, a RNA sequencing panel is designed with amplicons for detecting RNA fusion sequences. In various embodiments, a RNA sequencing panel includes cDNA amplicons that are derived from RNA transcripts. In various embodiments, step 210 involves designing amplicons with initial attributes for a DNA sequencing panel and involves designing amplicons with initial attributes for a RNA sequencing panel.

In various embodiments, the initial attributes of the amplicons are dictated by the target detection objective. For example, for amplicons of a DNA sequencing panel, the initial attributes of the amplicons are selected for particular gene loci of interest. As another example, for amplicons of a RNA sequencing panel, the initial attributes of the amplicons are selected for RNA sequences corresponding to gene loci of interest. As another example, for amplicons of a RNA sequencing panel, the initial attributes of the amplicons are selected for RNA fusion sequences corresponding to two gene loci of interest.

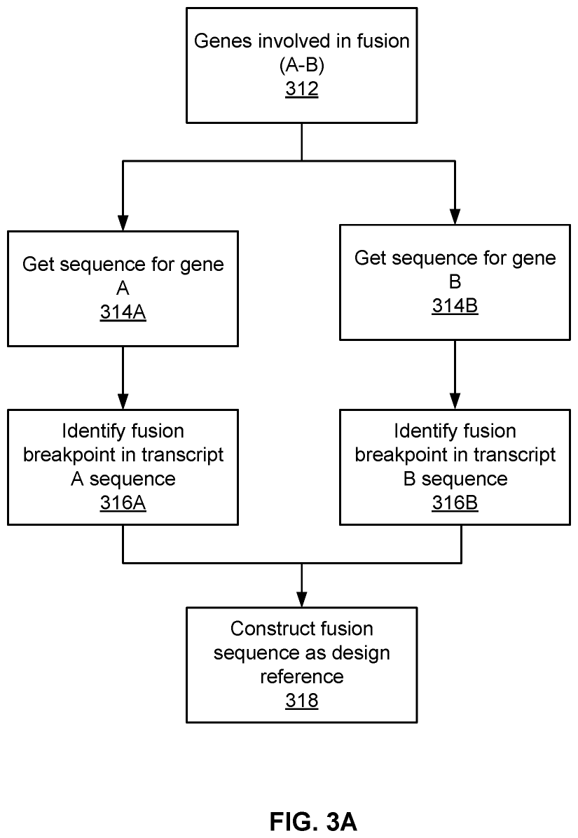

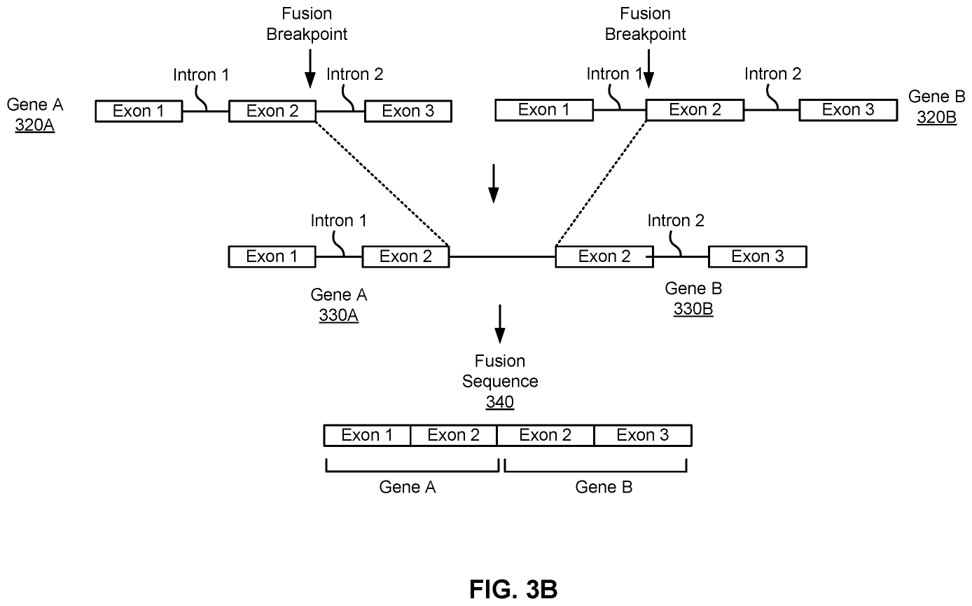

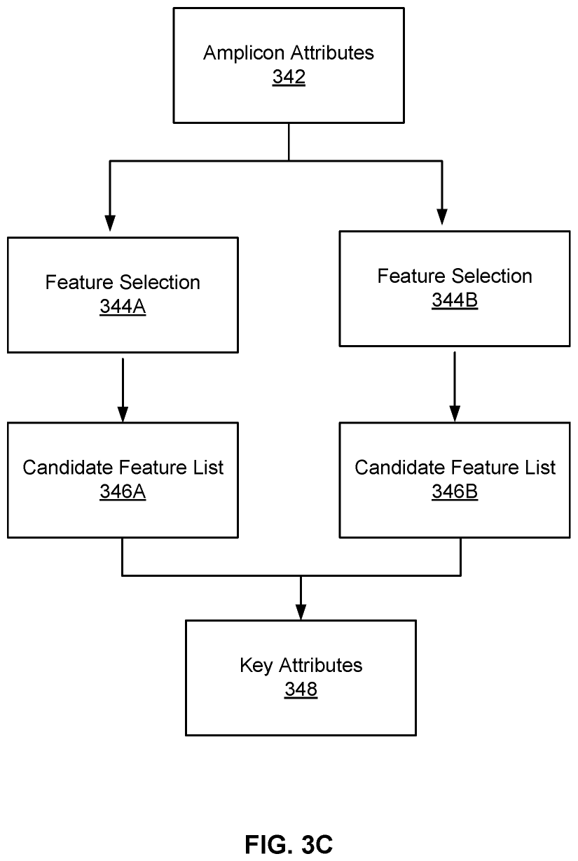

FIG. 3A depicts an example flow diagram for constructing a RNA fusion sequence, in accordance with an embodiment. Additional reference will be made to FIG. 3B, which depicts an example schematic for construction a fusion sequence, in accordance with an embodiment. Generally, the steps of constructing a RNA fusion sequence can be performed in step 210 (shown in FIG. 2) for generating amplicons with initial attributes.

As shown in FIG. 3A, step 312 involves identifying the genes involved in a particular fusion (e.g., gene A and gene B). As one example, the genes are involved in a fusion include BCR and ABL. At step 314 (e.g., step 314A and step 314B), sequences for gene A and gene B are obtained. For example, referring to FIG. 3B, sequences of gene A 320A and sequences of gene B 320B are obtained. Here, gene A 320A includes three exons and two introns. Similarly, gene B 320B includes three exons and two introns. In other embodiments, gene A and gene B can have additional or fewer introns/exons.

At step 316 (e.g., step 316A and 316B), the fusion breakpoint in gene A and fusion breakpoint in gene B are identified. For example, as shown in FIG. 3B, the fusion breakpoint for Gene A 320A is located between exon 2 and intron 2 of gene A. The fusion breakpoint for Gene B 320B is located between exon 2 and intron 1 of gene B.

At step 318, the fusion sequence is constructed as a design reference. Here, the fusion sequence can be an amplicon. In various embodiments, step 318 involves concatenating the sequence of gene A at the fusion breakpoint for gene A with the sequence of gene B at the fusion breakpoint for gene B, For example, as shown in FIG. 3B, the fusion breakpoints of gene A 320A and gene 330B are concatenated together (e.g., shown in the middle panel of FIG. 3B). In various embodiments, step 318 involves stitching together exon sequences of the first gene and the exon sequences of the second gene that flank the concatenated sequences at the fusion breakpoints. For example, the stitching together of exon sequences can involve removing introns from the two genes. For example, as shown in FIG. 3B, intron 1 in gene A 330A is removed and intron 2 of gene B 330B is removed. The fusion sequence 340 includes the two exons (e.g., exons 1 and 2) of gene A and two exons (e.g., exons 2 and exon 3) of gene B. Note, the exons 1 and 2 of gene A were originally flanking the fusion breakpoint identified for gene A. Additionally, the exons 2 and 3 of gene B were originally flanking the fusion breakpoint identified for gene B. Here, the junction between exon 2 of gene A and exon 2 of gene B represents the fusion point between the two genes. Given that the fusion sequence 340 does not include any intronic sequences, the fusion sequence 340 represents a RNA amplicon for inclusion in a RNA sequencing panel.

Returning to FIG. 2, step 220 involves determining amplicon performance of the amplicons with the initial attributes. Here, the amplicons with the initial attributes are used to sequence a target DNA (e.g., DNA derived from genomic DNA or cDNA derived from RNA transcript) and performance of the amplicons are recorded. The sequenced nucleic acids are then read.

In various embodiments, one or more data tables may be generated to quantify performance of each amplicon and its initial attributes. As an example, a data table is shown as Table 1 below. Here, table 1 represents an exemplary table of 600 amplicons tested against 20 attributes (e.g., attributes including primer length, AT %, GC %, etc.). It should be noted that TABLE 1 is exemplary and non-limiting. Different primary attributes may be selected for a desired application without departing from the disclosed principles. Additionally, in other embodiments, such a data table can be differently constructed with additional or fewer amplicons and/or additional or fewer attributes.

TABLE 1
Exemplary Primary Attribute Table

| Amp. | | | | | 20. |
|---|---|---|---|---|---|
| ID | 1. Primer length | 2. At % | 3. GC % | . . . | Performance |
| Amp. 1 | Primer length 1 | AT % 1 | GC % 1 | . . . | Performance 1 |
| Amp. 2 | Primer length 2 | AT % 2 | GC % 2 | . . . | Performance 2 |
| . . . | . . . | . . . | . . . | . . . | . . . |
| Amp. | Primer length | AT % 599 | GC % 599 | . . . | Performance |
| 599 | 599 | | | | 599 |
| Amp. | Primer length | AT % 600 | GC % 600 | . . . | Performance |
| 600 | 600 | | | | 600 |

At step 230, the tested amplicons are categorized into different categories depending on their performance Amplicon performance can include one or more of extent of coverage, panel uniformity, and normalized read value for the amplicon Amplicons are categorized into one of a plurality of categories that are indicative of the different performance of the amplicons. In one embodiment, amplicons are categorized into a low performer category, or a high performer category. In various embodiments, amplicons are categorized into a low performer category, an average performer category, and a higher performer category. In various embodiments, amplicons can be categorized into more than 3 categories that are indicative of the different performance of the amplicons.

Amplicon categorization can be implemented in different ways. In various embodiments, a benchmark or threshold is dynamically calculated using the average performance of all tested amplicons. Each tested amplicon is then compared in different criteria against the benchmark. As a result, each amplicon is then labeled with a metric to denote its performance against the known benchmark. In various embodiments, amplicons are divided up into the different categories depending on their performance. As an example, if amplicons are categorized into N different categories, the top 1/N % of amplicons are categorized into the top category, the next 1/N % of amplicons are categorized into the second category, and continuing all categories are filled.

In various embodiments, an additional step of normalization or read-count may be performed for each amplicon. The read-count can be normalized for each amplicon as a read percentage of each cell for example by dividing the read count of one amplicon to the total number of read counts of each cell.

In various embodiments, one or more of the categories of amplicons are selected. In some embodiments, one or more categories of amplicons are selected for training a prediction model, as shown in step 235 of FIG. 2. In various embodiments, the category of amplicons indicative of the highest performing amplicons is selected. For example, assuming there are three categories (e.g., low performers, average performers, and high performers), the high performer category of amplicons is selected. In various embodiments, the top 2 categories of amplicons including the highest performing amplicons are selected. In various embodiments, the top 3 categories of amplicons including the highest performing amplicons are selected. In various embodiments, the category including the lowest performing amplicons is selected. In various embodiments, the category including average performing amplicons is selected. In various embodiments, all categories are selected. Thus, the amplicons in the selected category or categories are used to train the prediction model. As an example, referring again exemplary Table 1, the initial attributes of the amplicons in the selected category or categories can be extracted from Table 1 and used to train the prediction model. Thus, the prediction model is trained to recognize patterns in attributes of high performing amplicons such that the prediction model can be deployed to predict whether other amplicons are likely to be high performers. In various embodiments, selected categories include all categories (and therefore, all amplicons). Thus, the prediction model is trained to recognize patterns in amplicon attributes that enable differentiation between differently performing amplicons. Thus, the prediction model can be deployed to predict the performance of other amplicons. Further details of the prediction model are described below.

In some embodiments, one or more categories of amplicons are selected to undergo feature selection at step 240 and/or step 250. In various embodiments, the category of amplicons indicative of the highest performing amplicons is selected. For example, assuming there are three categories (e.g., low performers, average performers, and high performers), the high performance category of amplicons is selected. In various embodiments, the top 2 categories of amplicons including the highest performing amplicons are selected. In various embodiments, the top 3 categories of amplicons including the highest performing amplicons are selected. In various embodiments, the category including the lowest performing amplicons is selected. In various embodiments, the category including average performing amplicons is selected. Thus, the amplicons in the selected categories can be analyzed in a feature selection process. As an example, referring again exemplary Table 1, the initial attributes of the amplicons in the selected category or categories can be extracted from Table 1 and analyzed in the subsequent feature selection process.

The next steps of feature selection (e.g., steps 240 and 250). In various embodiments, only one feature selection step is needed (e.g., steps 250 and 260 are not performed). In various embodiments, both feature selection steps are performed. Generally, the feature selection process(es) analyze the amplicons in the selected categories (selected in step 230) and identifies a subset of amplicon attributes, hereafter referred to as key attributes. Key attributes refer to amplicon attributes that are identified as particularly influential to the performance of amplicons. Therefore, if the selected categories include high performing amplicons, the feature selection process(es) identify key attributes that are particularly influential as to the high performance of the amplicons.

In various embodiments, feature selection at step 240 involves implementing one or more machine learned techniques. For example, machine learned techniques can involve implementing a ranking model involving a recursive feature elimination (RFE) process or a random forest classifier. Random Forest classifiers can involve a regression or tasks that operate by constructing a multitude of decision trees at training time and outputting the class that is the mode of the classes (classification) or mean prediction (regression) of the individual decision trees. A random forest classifier can measure feature importance based on Gini importance or Mean Decrease in Impurity (MDI) across the decision trees. As such, features (e.g., amplicon attributes) with the highest feature importance values (e.g., weights) can be selected through a machine-learned feature selection process.

In various embodiments, feature selection at step 240 involves implementing at least two feature selection processes. Reference is now made to FIG. 3C, which depicts an example flow diagram for performing a feature selection process to identify key attributes of amplicons, in accordance with an embodiment. Here, amplicon attributes 342 are analyzed under separate feature selection processes at steps 344A and 344B. In various embodiments, feature selection 344A refers to a recursive feature elimination (RFE) process. In various embodiments, feature selection 344B refers to implementation of a random forest classifier. Thus, the feature selection 344A results in the identification of a candidate feature list 346A and the feature selection 344B results in the identification of a candidate feature list 346B. Common attributes that are present in both candidate feature list 346A and candidate feature list 346B (e.g., attributes that are selected by both feature selection processes 344A and 344B) are identified as key attributes 348.

In various embodiments, the number of key attributes represents at least a 5-fold reduction in number of attributes in comparison to the number of amplicon attributes in the selected categories (e.g., selected at step 230). In various embodiments, the number of key attributes represents at least a 10-fold reduction in number of attributes in comparison to the number of amplicon attributes in the selected categories (e.g., selected at step 230). In various embodiments, the number of key attributes represents at least a 15-fold reduction in number of attributes in comparison to the number of amplicon attributes in the selected categories (e.g., selected at step 230). In various embodiments, the number of key attributes represents at least a 20-fold reduction in number of attributes in comparison to the number of amplicon attributes in the selected categories (e.g., selected at step 230). In various embodiments, the number of key attributes represents at least a 25-fold reduction, at least a 50-fold reduction, or at least 100-fold reduction in number of attributes in comparison to the number of amplicon attributes in the selected categories (e.g., selected at step 230).

In various embodiments, the total number of key attributes is at least 2 amplicon attributes. In various embodiments, the total number of key attributes is at least 3, at least 4, at least 5, at least 6, at least 7, at least 8, at least 9, at least 10, at least 11, at least 12, at least 13, at least 14, at least 15, at least 16, at least 17, at least 18, at least 19, or at least 20 amplicon attributes. In particular embodiments, the total number of key attributes is 3 attributes. In particular embodiments, the total number of key attributes is 5 attributes. In particular embodiments, the total number of key attributes is 8 attributes. In particular embodiments, the total number of key attributes is 10 attributes. In particular embodiments, the total number of key attributes is 12 attributes. In particular embodiments, the total number of key attributes is 15 attributes. In particular embodiments, the total number of key attributes is 18 attributes. In particular embodiments, the total number of key attributes is 20 attributes.

In the exemplary embodiment of Table 2 shown below, two key attributes (e.g., primer length and GC %) were identified from the twenty initial attributes shown in Table 1.

TABLE 2
Results of Correlation Study to Identify Significant Attributes

| Amp. ID | 1. Primer length | 2. GC % |
|---|---|---|
| Amp. 1 | Primer length 1 | GC % 1 |
| Amp. 2 | Primer length 2 | GC % 2 |
| . . . | | |
| Amp. 599 | Primer length 599 | GC % 599 |
| Amp. 600 | Primer length 600 | GC % 600 |

Returning to FIG. 2, at step 250, a second feature selection step may be performed. Here, the second feature selection step may be a correlation study. Correlation of numeric features are analyzed to identify and remove highly correlated features. Highly correlated attributes are those in which a change in one attribute causes a change in another attribute. The selection of the independent key attributes provides for a more precise selection of amplicons. In various embodiments, correlated features are defined as attributes with a correlation value above a threshold value. In various embodiments, the correlation value is between 0 and 1 and therefore, the threshold value can be a value of 0.2. In various embodiments, the threshold value is a value of 0.3. In various embodiments, the threshold value is a value of 0.4. In various embodiments, the threshold value is a value of 0.5. In various embodiments, the threshold value is a value of 0.55. In various embodiments, the threshold value is a value of 0.6. In various embodiments, the threshold value is a value of 0.65. In various embodiments, the threshold value is a value of 0.7. In various embodiments, the threshold value is a value of 0.75. In various embodiments, the threshold value is a value of 0.8. In various embodiments, the threshold value is a value of 0.85. In various embodiments, the threshold value is a value of 0.9. In various embodiments, the threshold value is a value of 0.95.

Step 260 involves a statistical analysis of the key attributes. In various embodiments, the statistical analysis can include calculation of statistical parameters. Example statistical parameters include mean, median, mode, range, and standard deviation. Thus, step 260 involves determining statistical parameters for the key attributes which were identified after the feature selection process(es).

The key attributes and/or the statistical parameters of the key attributes are used at step 270 to design new panels. Generally, improved amplicons are designed based on the key attributes. Thus, the improved amplicons may exhibit performance similar to the higher performing amplicons that were previously categorized (e.g., categorized at step 230). In various embodiments, improved amplicons are designed with key attributes with values that align with the statistical parameters of the key attributes. In one embodiment, a value of an attribute aligns with a statistical parameter of a key attribute if the value matches the statistical parameter. In various embodiments, a value of an attribute aligns with a statistical parameter of a key attribute if the value is within a certain percentage of the statistical parameter. As one example, the value of an attribute aligns with a statistical parameter of a key attribute if the value is within 10% of the statistical parameter of the key attribute. As one example, the value of an attribute aligns with a statistical parameter of a key attribute if the value is within 5% of the statistical parameter of the key attribute.

As an example, a statistical parameter of a key attribute may be a mean value of the key attribute. Thus, the improved amplicons are designed to align with the mean value of the key attribute. As another example, a statistical parameter of a key attribute may be a range of the key attribute. Thus, the improved amplicons are designed to have values of the key attribute that align with the range.

At step 280, new panels including the improved amplicons can be evaluated through a performance test. In various embodiments, the performance test includes sequencing the new panels and evaluating the performance of the new panels. Here, if the performance of the new panels exceeds a threshold performance metric, the design workflow process terminates. In various embodiments, if the new panels fail to meet the threshold performance metric, the design workflow process can revert to step 210 as shown by arrow and the designed amplicons can be. re-analyzed (e.g., through steps 210-270) to develop yet further improved amplicons.

In various embodiments, the performance test 280 involves deploying a prediction model to validate a panel including improved amplicons that are designed based on key attributes. Thus, the prediction model represents an in silico method of validating panels of improved amplicons after the improved amplicons have been designed using the amplicon design workflow. In various embodiments, the prediction model is prediction model 235 shown in FIG. 2. In various embodiments, deployment of the prediction model for in silico validation represents an alternative process to experimental validation of the panels including improved amplicons (e.g., actual sequencing of the improved amplicons and calculating performance metrics). In various embodiments, deployment of the prediction model for in silico validation represents a process in addition to experimental validation of the panels including improved amplicons (e.g., actual sequencing of the improved amplicons and calculating performance metrics). For example, the prediction model can be deployed to first generate an in silico prediction as to the performance of the panel. If the prediction indicates that the panel is likely to perform well, an experimental validation of the panel can be subsequently conducted to verify the predicted performance of the panel. Thus, an experimental validation need not be conducted for every validation of a new panel.

In various embodiments, the prediction model generates a prediction of the performance of the panel. Here, if the predicted performance of the new panels exceeds a threshold performance metric, the process terminates at step 290. In various embodiments, if the new panels fail to meet the threshold performance metric, the process can revert to step 210 as shown by arrow.

In various embodiments, the threshold performance metric is a threshold panel uniformity. In various embodiments, the threshold panel uniformity metric is at least 70%. In various embodiments, the threshold panel uniformity metric is at least 70%. In various embodiments, the threshold panel uniformity metric is at least 80%. In various embodiments, the threshold panel uniformity metric is at least 85%. In various embodiments, the threshold panel uniformity metric is at least 86%, at least 87%, at least 88%, at least 89%, at least 90%, at least 91%, at least 92%, at least 93%, at least 94%, at least 95%, at least 96%, at least 97%, at least 98%, or at least 99%.

In various embodiments, the threshold performance metric is a sensitivity of at least 70%. In various embodiments, the threshold performance metric is a sensitivity of at least 80%. In various embodiments, the threshold performance metric is a sensitivity of at least 85%. In various embodiments, the threshold performance metric is a sensitivity of at least 86%, at least 87%, at least 88%, at least 89%, at least 90%, at least 91%, at least 92%, at least 93%, at least 94%, at least 95%, at least 96%, at least 97%, at least 98%, or at least 99%. Here, sensitivity refers to the true positives divided by the total real positives.

In various embodiments, the threshold performance metric is a specificity of at least 70%. In various embodiments, the threshold performance metric is a specificity of at least 80%. In various embodiments, the threshold performance metric is a specificity of at least 85%. In various embodiments, the threshold performance metric is a specificity of at least 86%, at least 87%, at least 88%, at least 89%, at least 90%, at least 91%, at least 92%, at least 93%, at least 94%, at least 95%, at least 96%, at least 97%, at least 98%, or at least 99%. Here, specificity refers to the true negatives divided by the total real negatives.

### Example Amplicon Attributes

Embodiments described herein refer to amplicon attributes. In various embodiments, amplicon attributes refer to initial attributes of amplicons (e.g., amplicons with initial attributes at step 210 in FIG. 2). Thus, initial attributes of the amplicons can be analyzed using the amplicon design workflow to identify key attributes of the amplicons. In various embodiments, key attributes of amplicons refer to attributes of amplicons that are identified through a feature selection process as attributes that likely lead to high performance amplicons. Thus, key attributes can be used to design improved amplicons that likely exhibit high performance

In various embodiments, amplicon attributes refer to characteristics of primers that target the amplicon (e.g., primers that enable nucleic acid amplification of the amplicon). For example, the primers can be a forward and reverse primer pair that hybridize with regions of the amplicon, thereby enabling extension of nucleic acid strands along the amplicon sequence. In various embodiments, amplicon attributes refer to characteristics of the amplicon, including but not limited to the characteristics of the insert, which is the region of interest amplified by primers. In various embodiments, amplicon attributes include both characteristics of amplicons and characteristics of primers that target the amplicon.

In various embodiments, amplicon attributes may include amplicon length, secondary structure prediction, primer specificity, amplicon GC, primer length, percentage of GC content in primer, GC content at 3′ end of primer, GC content at 5′ end of primer, number of G or C bases within the last five bases of 3′ end, stability for the last five 3′ bases in primer (measured by maximum dG—Gibbs Free Energy—for disruption the structure), number of unknown bases in primer, number of ambiguous bases in primer, ambiguity code for ambiguous bases, long runs of single base in primer, number of tandem repeats in primer, number of dinucleotide repeats in primer, position of dinucleotide repeats in primer, number of trinucleotide repeats in primer, position of trinucleotide repeats in primer, number of tetranucleotide repeats in primer, position of tetranucleotide repeats in primer, number of pentanucleotide repeats in primer, position of pentanucleotide repeats in primer, number of hexanucleotide repeats in primer, position of hexanucleotide repeats in primer, primer melting temperature, melting temperature difference between forward and reverse primers, number of inverted repeats in primer, length of inverted repeats in primer, percentage of GC content in inverted repeats in primer, number of primer secondary hairpin structure, dG value of primer secondary hairpin structure, in-silico melting temperature of predicted primer secondary hairpin structure, primer self-dimer folding dG value, in-silico melting temperature of predicted primer self-dimer folding, primer pair heterodimer (cross dimers), primer pair heterodimer folding dG value, primer pair heterodimer melting temperature, number of primer heterodimers in a pool of primers, folding dG value for all in-silico predicted heterodimers, in-silico melting temperature of all in-silico predicted primer heterodimers, number of primer mispriming sites in template library, number of primer mispriming site in a pool of amplicons, number of primer priming sites with no mismatch in last 10 bases of 3′ end, number of primer priming sites with no mismatch in last 3 bases of 3′ end, number of primer priming sites with 1 mismatch in last 10 bases of 3′ end, number of primer priming sites with 1 mismatch in last 3 bases of 3′ end, number of primer priming sites with 1 mismatch in last 5 bases of 3′ end, number of primer priming sites with 2 mismatch in last 10 bases of 3′ end, number of primer priming sites with 2 mismatch in last 3 bases of 3′ end, number of primer priming sites with 2 mismatch in last 10 bases of 3′ end, number of primer priming sites with 2 mismatch in last 3 bases of 3′ end, number of primer priming sites with 1 mismatch in last 5 bases of 3′ end, number of SNP (single nucleotide polymorphisms) in primer, number of common SNP (>1%) in primer, number of one nucleotide substitution SNP in primer, position of one nucleotide substitution SNP in primer, number of one nucleotide deletion SNP in primer, position of one nucleotide deletion SNP in primer, number of one nucleotide insertion SNP in primer, position of one nucleotide insertion SNP in primer, amplicon length, percentage of GC content in amplicon, melting temperature of amplicon, insert length, percentage of GC content in insert, melting temperature of insert, percentage of GC content in first 100 bp in 5′ end of amplicon, melting temperature of first 100 bp in 5′ end of amplicon, percentage of GC content in last 150 bp in 3′ end of amplicon, melting temperature of last 150 bp in 5′ end of amplicon, target position to the 5′ end of amplicon, target position to the 3′ end of amplicon, target position to the 5′ end of insert, target position to the 3′ end of insert, bases of target inside forward primer, bases of target inside reverse primer, number of homopolymer runs in amplicon, length of homopolymer A runs in amplicon, position of homopolymer A in amplicon, length of homopolymer T runs in amplicon, position of homopolymer T in amplicon, length of homopolymer C runs in amplicon, position of homopolymer C in amplicon, length of homopolymer G runs in amplicon, position of homopolymer G in amplicon, number of tandem repeats in amplicon, number of dinucleotide repeats in amplicon, position of dinucleotide repeats in amplicon, number of trinucleotide repeats in amplicon, position of trinucleotide repeats in amplicon, number of tetranucleotide repeats in amplicon, position of tetranucleotide repeats in amplicon, number of pentanucleotide repeats in amplicon, position of pentanucleotide repeats in amplicon, number of hexanucleotide repeats in amplicon, position of hexanucleotide repeats in amplicon, target position to the homopolymers, target position to the tandem repeats, number of common SNP in amplicon, position of common SNP in amplicon, number of common SNP in insert, position of common SNP in insert, target position to common SNPs, insert specificity in designed genome, the minimal sequencing quality allowed for primer, the minimal sequencing quality allowed for 3′ end last five bases of primer, space between amplicons, maximum overlapping bases allowed for amplicons. It should be noted that the amplicon attributes described herein are exemplary and other amplicon attributes may be used without deviating from the disclosed principles.

### Example Panels

Panels described herein refer to groups of amplicons that can be sequenced to build a sequencing library. In various embodiments, a panel is a DNA panel including DNA amplicons for building DNA libraries. In various embodiments, a panel is a RNA panel including RNA amplicons for building RNA libraries. In various embodiments, a RNA panel includes RNA amplicons designed for RNA fusion transcripts. Thus, implementation of the RNA transcript enables building a RNA library that detects one or more RNA fusion transcripts.

In various embodiments, a panel can include 2 amplicons. In various embodiments, a panel can include 5 amplicons. In various embodiments, a panel can include 10 amplicons. In various embodiments, a panel can include 20 amplicons. In various embodiments, a panel can include 50 amplicons. In various embodiments, a panel can include 100 amplicons. In various embodiments, a panel can include 200 amplicons. In various embodiments, a panel can include 300 amplicons. In various embodiments, a panel can include 400 amplicons. In various embodiments, a panel can include 500 amplicons. In various embodiments, a panel can include 600 amplicons. In various embodiments, a panel can include 700 amplicons. In various embodiments, a panel can include 800 amplicons. In various embodiments, a panel can include 900 amplicons. In various embodiments, a panel can include 1000 amplicons.

In various embodiments, a panel can include at least 2 amplicons. In various embodiments, a panel can include at least 5 amplicons. In various embodiments, a panel can include at least 10 amplicons. In various embodiments, a panel can include at least 20 amplicons. In various embodiments, a panel can include at least 50 amplicons. In various embodiments, a panel can include at least 100 amplicons. In various embodiments, a panel can include at least 200 amplicons. In various embodiments, a panel can include at least 300 amplicons. In various embodiments, a panel can include at least 400 amplicons. In various embodiments, a panel can include at least 500 amplicons. In various embodiments, a panel can include at least 600 amplicons. In various embodiments, a panel can include at least 700 amplicons. In various embodiments, a panel can include at least 800 amplicons. In various embodiments, a panel can include at least 900 amplicons. In various embodiments, a panel can include at least 1000 amplicons.

In various embodiments, a panel can include between 5 and 1000 amplicons. In various embodiments, a panel can include between 20 and 800 amplicons. In various embodiments, a panel can include between 50 and 600 amplicons. In various embodiments, a panel can include between 100 and 500 amplicons. In various embodiments, a panel can include between 200 and 400 amplicons. In various embodiments, a panel can include between 250 and 300 amplicons. In various embodiments, a panel can include between 100 and 1000 amplicons. In various embodiments, a panel can include between 200 and 1000 amplicons. In various embodiments, a panel can include between 300 and 1000 amplicons. In various embodiments, a panel can include between 400 and 1000 amplicons. In various embodiments, a panel can include between 500 and 1000 amplicons. In various embodiments, a panel can include between 600 and 1000 amplicons. In various embodiments, a panel can include between 700 and 1000 amplicons. In various embodiments, a panel can include between 800 and 1000 amplicons. In various embodiments, a panel can include between 900 and 1000 amplicons. In various embodiments, a panel can include between 10 and 500 amplicons. In various embodiments, a panel can include between 10 and 250 amplicons. In various embodiments, a panel can include between 10 and 150 amplicons. In various embodiments, a panel can include between 10 and 100 amplicons. In various embodiments, a panel can include between 10 and 75 amplicons. In various embodiments, a panel can include between 10 and 50 amplicons. In various embodiments, a panel can include between 100 and 500 amplicons. In various embodiments, a panel can include between 120 and 450 amplicons. In various embodiments, a panel can include between 150 and 400 amplicons. In various embodiments, a panel can include between 180 and 300 amplicons. In various embodiments, a panel can include between 200 and 250 amplicons.

In various embodiments, a panel can include amplicons with initial attributes. Such a panel includes amplicons that were not designed using the amplicon design workflow described herein. For example a panel including amplicons with initial attributes is found at step 210 of FIG. 2. Following implementation of the amplicon design workflow, a panel including improved amplicons can be generated. Here, the improved amplicons are designed based on key attributes of amplicons that are identified (e.g., through a feature selection process) in the amplicon design workflow. Thus, the panel including improved amplicons designed based on key attributes, when implemented, exhibits improved performance in comparison to a panel including amplicons with initial attributes.

In various embodiments, the panel including improved amplicons achieves a panel uniformity of at least 70%. In various embodiments, the panel including improved amplicons achieves a panel uniformity of at least 80%. In various embodiments, the panel including improved amplicons achieves a panel uniformity of at least 85%. In various embodiments, the panel including improved amplicons achieves a panel uniformity of at least 86%, at least 87%, at least 88%, at least 89%, at least 90%, at least 91%, at least 92%, at least 93%, at least 94%, at least 95%, at least 96%, at least 97%, at least 98%, or at least 99%.

In various embodiments, the panel includes improved RNA fusion amplicons. In such embodiments, the panel including improved RNA fusion amplicons can achieve improved detection of the presence of RNA fusions in single cells. For example, a single cell can be called as having a RNA fusion based a threshold of M reads per cell per fusion transcript. In various embodiments, M is 20 reads. In various embodiments, M is 5, 6, 7, 8, 9, 10, 11, 12, 13, 14, 15, 16, 17, 18, or 19 reads. In various embodiments, M is 21, 22, 23, 24, 25, 26, 27, 28, 29, 30, 31, 32, 33, 34, 35, 36, 37, 38, 39, or 40 reads.

In various embodiments, the panel including improved RNA fusion amplicons can achieve a sensitivity of at least 70%. In various embodiments, the panel including improved RNA fusion amplicons can achieve a sensitivity of at least 80%. In various embodiments, the panel including improved RNA fusion amplicons can achieve a sensitivity of at least 85%. In various embodiments, the panel including improved RNA fusion amplicons can achieve a sensitivity of at least 86%, at least 87%, at least 88%, at least 89%, at least 90%, at least 91%, at least 92%, at least 93%, at least 94%, at least 95%, at least 96%, at least 97%, at least 98%, or at least 99%. Here, sensitivity refers to the true positives divided by the total real positives.

In various embodiments, the panel including improved RNA fusion amplicons can achieve a specificity of at least 70%. In various embodiments, the panel including improved RNA fusion amplicons can achieve a specificity of at least 80%. In various embodiments, the panel including improved RNA fusion amplicons can achieve a specificity of at least 85%. In various embodiments, the panel including improved RNA fusion amplicons can achieve a specificity of at least 86%, at least 87%, at least 88%, at least 89%, at least 90%, at least 91%, at least 92%, at least 93%, at least 94%, at least 95%, at least 96%, at least 97%, at least 98%, or at least 99%. Here, specificity refers to the true negatives divided by the total real negatives.

### Example Prediction Model

Embodiments described herein refer to the generation of a prediction model. As one example, the prediction model can be prediction model 235 shown in FIG. 2 of the amplicon design workflow. In various embodiments, the prediction model is deployed during the performance test at step 280 of FIG. 2. Therefore, the prediction model can be used to validate a new panel with amplicons that have been designed using the amplicon design workflow.

Generally, a prediction model is structured such that it analyzes amplicon attributes (e.g., amplicon features) of a panel of amplicons and generates a predicted performance for the panel of amplicons. For example, the prediction model can generate a prediction of panel uniformity based on the attributes of amplicons in a panel. In such scenarios, deployment of the prediction model on a panel of amplicons is useful for predicting whether the panel is likely to exhibit high performance according to a predicted panel uniformity measurement.

In various embodiments, the prediction model is any one of a regression model (e.g., linear regression, logistic regression, or polynomial regression), decision tree, random forest, gradient boosted machine learning model, support vector machine, Naive Bayes model, k-means cluster, or neural network (e.g., feed-forward networks, convolutional neural networks (CNN), deep neural networks (DNN), autoencoder neural networks, generative adversarial networks, or recurrent networks (e.g., long short-term memory networks (LSTM), bi-directional recurrent networks, deep bi-directional recurrent networks), or any combination thereof. In particular embodiments, the prediction model is support vector classifier (SVC). In particular embodiments, the prediction model is a random forest classifier. In particular embodiments, the prediction model is a K Neighbors Classifier (KNC).

The prediction model can be trained using a machine learning implemented method, such as any one of a linear regression algorithm, logistic regression algorithm, decision tree algorithm, support vector machine classification, Naïve Bayes classification, K-Nearest Neighbor classification, random forest algorithm, deep learning algorithm, gradient boosting algorithm, and dimensionality reduction techniques such as manifold learning, principal component analysis, factor analysis, autoencoder regularization, and independent component analysis, or combinations thereof. In particular embodiments, the machine learning implemented method is a logistic regression algorithm In particular embodiments, the machine learning implemented method is a random forest algorithm In particular embodiments, the machine learning implemented method is a gradient boosting algorithm, such as XGboost. In various embodiments, the prediction model is trained using supervised learning algorithms, unsupervised learning algorithms, semi-supervised learning algorithms (e.g., partial supervision), weak supervision, transfer, multi-task learning, or any combination thereof.

In various embodiments, the prediction model has one or more parameters, such as hyperparameters or model parameters. Hyperparameters are generally established prior to training. Examples of hyperparameters include the learning rate, depth or leaves of a decision tree, number of hidden layers in a deep neural network, number of clusters in a k-means cluster, penalty in a regression model, and a regularization parameter associated with a cost function. Model parameters are generally adjusted during training. Examples of model parameters include weights associated with nodes in layers of neural network, support vectors in a support vector machine, node values in a decision tree, and coefficients in a regression model. The model parameters of the prediction model are trained (e.g., adjusted) using the training data to improve the predictive capacity of the prediction model.

Generally, the prediction model is trained using training data. In various embodiments, the training data includes one or more panels including amplicons with attributes. In various embodiments, the training data can include ground truth labels. For example, for amplicons in the one or more panels in the training data, the training data can include labels that indicate a performance of the amplicon. In various embodiments, amplicons are labeled in one of a plurality of categories that are indicative of the performance of the amplicon. As one example, the plurality of categories can include 1) low performance amplicons, 2) average performance amplicons, and 3) high performance amplicons. Thus, over training iterations, the prediction model is trained to predict attributes that likely lead to different categories of amplicon performances. Therefore when the prediction model is deployed, the prediction model can analyze attributes of amplicons of a panel and categorize the amplicons in one of the plurality of categories.

In various embodiments, the training data can be obtained from a split of a dataset. For example, the dataset can undergo a 50:50 training:testing dataset split. In some embodiments, the dataset can undergo a 60:40 training:testing dataset split. In some embodiments, the dataset can undergo a 70:30 training:testing dataset split. In some embodiments, the dataset can undergo a 80:20 training:testing dataset split.

### Example Cancers

Embodiments described herein refer to conducting cellular analysis on one or more cells for purposes characterizing cancers at the single cell level. For example, the amplicon design workflow can be implemented to design panels (e.g., DNA panels or RNA panels) for detecting nucleic acid events (e.g., DNA mutations, RNA fusion events). As such, the presence or absence of nucleic acid events in genomic DNA or in RNA transcripts can be indicative of a form of cancer. Thus, single cell analysis using panels including improved amplicons that have been generated using the amplicon design workflow can reveal characteristics of cancer in single cells or populations of cells.

In various embodiments, the methods disclosed herein are useful for characterizing a wide variety of caners, including but not limited to the following: Acute Lymphoblastic Leukemia (ALL), Acute Myeloid Leukemia (AML), Adrenocortical Carcinoma, AIDS-Related Cancers, Kaposi Sarcoma (Soft Tissue Sarcoma), AIDS-Related Lymphoma (Lymphoma), Primary CNS Lymphoma (Lymphoma), Anal Cancer, Astrocytomas, Atypical Teratoid/Rhabdoid Tumor, Childhood, Central Nervous System (Brain Cancer), Basal Cell Carcinoma, Bile Duct Cancer, Bladder Cancer. Childhood Bladder Cancer, Bone Cancer (includes Ewing Sarcoma and Osteosarcoma and Malignant Fibrous Histiocytoma), Brain Tumors, Breast Cancer, Childhood Breast Cancer, Bronchial Tumors, Burkitt Lymphoma (Non-Hodgkin Lymphoma, Carcinoid Tumor (Gastrointestinal), Childhood Carcinoid Tumors, Cardiac (Heart) Tumors, Central Nervous System tumors. Atypical Teratoid/Rhabdoid Tumor, Childhood (Brain Cancer), Embryonal Tumors, Childhood (Brain Cancer), Germ Cell Tumor (Childhood Brain Cancer), Primary CNS Lymphoma, Cervical Cancer, Childhood Cervical Cancer, Cholangiocarcinoma, Chordoma (Childhood), Chronic Lymphocytic Leukemia (CLL), Chronic Myelogenous Leukemia (CML), Chronic Myeloproliferative Neoplasms, Colorectal Cancer, Childhood Colorectal Cancer, Craniopharyngioma (Childhood Brain Cancer), Cutaneous T-Cell Lymphoma, Ductal Carcinoma In Situ (DCIS), Embryonal Tumors, (Childhood Brain CNS Cancers), Endometrial Cancer (Uterine Cancer), Ependymoma, Esophageal Cancer, Childhood Esophageal Cancer, Esthesioneuroblastoma (Head and Neck Cancer), Ewing Sarcoma (Bone Cancer), Extracranial Germ Cell Tumors, Extragonadal Germ Cell Tumors, Eye Cancer, Childhood Intraocular Melanoma, Intraocular Melanoma, Retinoblastoma, Fallopian Tube Cancer, Fibrous Histiocytoma of Bone (Malignant, and Osteosarcoma), Gallbladder Cancer, Gastric (Stomach) Cancer, Childhood Gastric (Stomach) Cancer, Gastrointestinal Carcinoid Tumor, Gastrointestinal Stromal Tumors (GIST) (Soft Tissue Sarcoma), Childhood Gastrointestinal Stromal Tumors, Germ Cell Tumors, Childhood Central Nervous System Germ Cell Tumors, Childhood Extracranial Germ Cell Tumors, Extragonadal Germ Cell Tumors, Ovarian Germ Cell Tumors, Testicular Cancer, Gestational Trophoblastic Disease, Hairy Cell Leukemia, Head and Neck Cancer, Heart Tumors, Hepatocellular (Liver) Cancer, Histiocytosis (Langerhans Cell Cancer), Hodgkin Lymphoma, Hypopharyngeal Cancer (Head and Neck Cancer), Intraocular Melanoma, Childhood Intraocular Melanoma, Islet Cell Tumors, (Pancreatic Neuroendocrine Tumors), Kaposi Sarcoma (Soft Tissue Sarcoma), Kidney (Renal Cell) Cancer, Langerhans Cell Histiocytosis, Laryngeal Cancer (Head and Neck Cancer), Leukemia, Lip and Oral Cavity Cancer (Head and Neck Cancer), Liver Cancer, Lung Cancer (Non-Small Cell and Small Cell), Childhood Lung Cancer, Lymphoma, Male Breast Cancer, Malignant Fibrous Histiocytoma of Bone and Osteosarcoma, Melanoma, Childhood Melanoma, Melanoma (Intraocular Eye), Childhood Intraocular Melanoma, Merkel Cell Carcinoma (Skin Cancer), Mesothelioma, Childhood Mesothelioma, Metastatic Cancer, Metastatic Squamous Neck Cancer with Occult Primary (Head and Neck Cancer), Midline Tract Carcinoma With NUT Gene Changes, Mouth Cancer (Head and Neck Cancer), Multiple Endocrine Neoplasia Syndromes—see Unusual Cancers of Childhood, Multiple Myeloma/Plasma Cell Neoplasms, Mycosis Fungoides (Lymphoma), Myelodysplastic Syndromes, Myelodysplastic/Myeloproliferative Neoplasms, Myelogenous Leukemia, Chronic (CML), Myeloid Leukemia, (Acute AML), Myeloproliferative Neoplasms, Nasal Cavity and Paranasal Sinus Cancer (Head and Neck Cancer), Nasopharyngeal Cancer (Head and Neck Cancer), Neuroblastoma, Non-Hodgkin Lymphoma, Non-Small Cell Lung Cancer, Oral Cancer (Lip and Oral Cavity Cancer and Oropharyngeal Cancer), Osteosarcoma and Malignant Fibrous Histiocytoma of Bone, Ovarian Cancer, Childhood Ovarian Cancer, Pancreatic Cancer, Childhood Pancreatic Cancer, Pancreatic Neuroendocrine Tumors (Islet Cell Tumors), Papillomatosis, Paraganglioma, Childhood Paraganglioma, Paranasal Sinus and Nasal Cavity Cancer, Parathyroid Cancer, Penile Cancer, Pharyngeal Cancer, Pheochromocytoma, Childhood Pheochromocytoma, Pituitary Tumor, Plasma Cell Neoplasm/Multiple Myeloma, Pleuropulmonary Blastoma, Pregnancy and Breast Cancer, Primary Central Nervous System (CNS) Lymphoma, Primary Peritoneal Cancer, Prostate Cancer, Rectal Cancer, Recurrent Cancer, Renal Cell (Kidney) Cancer, Retinoblastoma, Rhabdomyosarcoma, Salivary Gland Cancer, Sarcoma, Childhood Rhabdomyosarcoma (Soft Tissue Sarcoma), Childhood Vascular Tumors (Soft Tissue Sarcoma), Ewing Sarcoma (Bone Cancer), Kaposi Sarcoma (Soft Tissue Sarcoma), Osteosarcoma (Bone Cancer), Soft Tissue Sarcoma, Uterine Sarcoma, Sézary Syndrome (Lymphoma), Skin Cancer, Childhood Skin Cancer, Small Cell Lung Cancer, Small Intestine Cancer, Soft Tissue Sarcoma, Squamous Cell Carcinoma of the Skin, Squamous Neck Cancer with Occult Primary, Stomach (Gastric) Cancer, Childhood Stomach, T-Cell Lymphoma, Testicular Cancer, Childhood Testicular Cancer, Throat Cancer, Nasopharyngeal Cancer, Oropharyngeal Cancer, Hypopharyngeal Cancer, Thymoma and Thymic Carcinoma, Thyroid Cancer, Transitional Cell Cancer of the Renal Pelvis and Ureter Kidney (Renal Cell Cancer), Ureter and Renal Pelvis (Transitional Cell Cancer Kidney Renal Cell Cancer), Urethral Cancer, Uterine Cancer (Endometrial), Uterine Sarcoma, Vaginal Cancer, Childhood Vaginal Cancer, Vascular Tumors (Soft Tissue Sarcoma), Vulvar Cancer, Wilms Tumor (and Other Childhood Kidney Tumors).

### Nucleic Acid Amplification

Embodiments disclosed herein involve performing a nucleic acid amplification reaction. For example, a nucleic acid amplification reaction can be performed to generate amplicons for sequencing. Thus, the amplicon performance and/or panel performance can be evaluated.

Generally, a nucleic acid amplification reaction for generating amplicons can involve the use of primers. Such primers can be designed to hybridize with regions of the amplicons and therefore, the appropriate nucleic acid extension can proceed off of the hybridized primer. In various embodiments, primers can include gene specific primers. For example, gene specific primers can include a forward and reverse primer pair that targets a genomic locus of a specific gene of interest. In various embodiments, primers can include universal primers. For example, universal primers can include an oligodT primer that hybridizes with a polyA tail of a RNA transcript. In various embodiments, primers can include random primers. For example, random primers can be designed to target a region of a nucleic acid, such as a cDNA sequence that has been reverse transcribed from a RNA transcript. Therefore, nucleic acid amplification can proceed off of the hybridized random primer. As described herein, primers for nucleic acid amplification have characteristics, which may also be referred to as attributes of the amplicons (e.g., amplicon attributes) that the primers target.

In various embodiments, primers are part of a primer set for the amplification of a target nucleic acid, the primer set including a forward primer and a reverse primer that are complementary to a target nucleic acid or the complement thereof. In some embodiments, amplification can be performed using multiple target-specific primer pairs in a single amplification reaction, wherein each primer pair includes a forward target-specific primer and a reverse target-specific primer, where each includes at least one sequence that substantially complementary or substantially identical to a corresponding target sequence in the sample, and each primer pair having a different corresponding target sequence. Accordingly, certain methods herein are used to detect or identify multiple target sequences from a single cell.

In various embodiments, primers may contain primers for one or more nucleic acid of interest, e.g. one or more genes of interest. The number of primers for genes of interest that are added may be from about one to 500, e.g., about 1 to 10 primers, about 10 to 20 primers, about 20 to 30 primers, about 30 to 40 primers, about 40 to 50 primers, about 50 to 60 primers, about 60 to 70 primers, about 70 to 80 primers, about 80 to 90 primers, about 90 to 100 primers, about 100 to 150 primers, about 150 to 200 primers, about 200 to 250 primers, about 250 to 300 primers, about 300 to 350 primers, about 350 to 400 primers, about 400 to 450 primers, about 450 to 500 primers, or about 500 primers or more.

In various embodiments, primers and/or reagents may be added to a discrete entity, e.g., a microdroplet, in one step, or in more than one step. For instance, the primers may be added in two or more steps, three or more steps, four or more steps, or five or more steps. Regardless of whether the primers are added in one step or in more than one step, they may be added after the addition of a lysing agent, prior to the addition of a lysing agent, or concomitantly with the addition of a lysing agent. When added before or after the addition of a lysing agent, the PCR primers may be added in a separate step from the addition of a lysing agent. In some embodiments, the discrete entity, e.g., a microdroplet, may be subjected to a dilution step and/or enzyme inactivation step prior to the addition of the PCR reagents. Exemplary embodiments of such methods are described in PCT Publication No. WO 2014/028378, the disclosure of which is incorporated by reference herein in its entirety and for all purposes.

Primers and oligonucleotides used in embodiments herein comprise nucleotides. A nucleotide comprises any compound, including without limitation any naturally occurring nucleotide or analog thereof, which can bind selectively to, or can be polymerized by, a polymerase. Typically, but not necessarily, selective binding of the nucleotide to the polymerase is followed by polymerization of the nucleotide into a nucleic acid strand by the polymerase; occasionally however the nucleotide may dissociate from the polymerase without becoming incorporated into the nucleic acid strand, an event referred to herein as a “non-productive” event. Such nucleotides include not only naturally occurring nucleotides but also any analogs, regardless of their structure, that can bind selectively to, or can be polymerized by, a polymerase. While naturally occurring nucleotides typically comprise base, sugar and phosphate moieties, the nucleotides of the present disclosure can include compounds lacking any one, some or all of such moieties. For example, the nucleotide can optionally include a chain of phosphorus atoms comprising three, four, five, six, seven, eight, nine, ten or more phosphorus atoms. In some embodiments, the phosphorus chain can be attached to any carbon of a sugar ring, such as the 5′ carbon. The phosphorus chain can be linked to the sugar with an intervening O or S. In one embodiment, one or more phosphorus atoms in the chain can be part of a phosphate group having P and O. In another embodiment, the phosphorus atoms in the chain can be linked together with intervening O, NH, S, methylene, substituted methylene, ethylene, substituted ethylene, CNH₂, C(O), C(CH₂), CH₂CH₂, or C(OH)CH₂R (where R can be a 4-pyridine or 1-imidazole). In one embodiment, the phosphorus atoms in the chain can have side groups having O, BH3, or S. In the phosphorus chain, a phosphorus atom with a side group other than O can be a substituted phosphate group. In the phosphorus chain, phosphorus atoms with an intervening atom other than O can be a substituted phosphate group. Some examples of nucleotide analogs are described in Xu, U.S. Pat. No. 7,405,281.

In some embodiments, the nucleotide comprises a label and referred to herein as a “labeled nucleotide”; the label of the labeled nucleotide is referred to herein as a “nucleotide label”. In some embodiments, the label can be in the form of a fluorescent moiety (e.g. dye), luminescent moiety, or the like attached to the terminal phosphate group, i.e., the phosphate group most distal from the sugar. Some examples of nucleotides that can be used in the disclosed methods and compositions include, but are not limited to, ribonucleotides, deoxyribonucleotides, modified ribonucleotides, modified deoxyribonucleotides, ribonucleotide polyphosphates, deoxyribonucleotide polyphosphates, modified ribonucleotide polyphosphates, modified deoxyribonucleotide polyphosphates, peptide nucleotides, modified peptide nucleotides, metallonucleosides, phosphonate nucleosides, and modified phosphate-sugar backbone nucleotides, analogs, derivatives, or variants of the foregoing compounds, and the like. In some embodiments, the nucleotide can comprise non-oxygen moieties such as, for example, thio- or borano-moieties, in place of the oxygen moiety bridging the alpha phosphate and the sugar of the nucleotide, or the alpha and beta phosphates of the nucleotide, or the beta and gamma phosphates of the nucleotide, or between any other two phosphates of the nucleotide, or any combination thereof. “Nucleotide 5′-triphosphate” refers to a nucleotide with a triphosphate ester group at the 5′ position, and is sometimes denoted as “NTP”, or “dNTP” and “ddNTP” to particularly point out the structural features of the ribose sugar. The triphosphate ester group can include sulfur substitutions for the various oxygens, e.g. α-thio-nucleotide 5′-triphosphates. For a review of nucleic acid chemistry, see: Shabarova, Z. and Bogdanov, A. Advanced Organic Chemistry of Nucleic Acids, VCH, New York, 1994.

Any nucleic acid amplification method may be utilized, such as a PCR-based assay, e.g., quantitative PCR (qPCR), or an isothermal amplification may be used to detect the presence of certain nucleic acids, e.g., genes, of interest, present in discrete entities or one or more components thereof, e.g., cells encapsulated therein. In various embodiments, nucleic acid amplification can be performed in discrete entities within a microfluidic device or a portion thereof or any other suitable location. The conditions of such amplification or PCR-based assays may include detecting nucleic acid amplification over time and may vary in one or more ways.

One or both primers of a primer set may comprise a barcode sequence described herein. In some embodiments, individual cells, for example, are isolated in discrete entities, e.g., droplets. These cells may be lysed and their nucleic acids barcoded. This process can be performed on a large number of single cells in discrete entities with unique barcode sequences enabling subsequent deconvolution of mixed sequence reads by barcode to obtain single cell information. This approach provides a way to group together nucleic acids originating from large numbers of single cells. Additionally, affinity reagents such as antibodies can be conjugated with nucleic acid labels, e.g., oligonucleotides including barcodes, which can be used to identify antibody type, e.g., the target specificity of an antibody. These reagents can then be used to bind to the proteins within or on cells, thereby associating the nucleic acids carried by the affinity reagents to the cells to which they are bound. These cells can then be processed through a barcoding workflow as described herein to attach barcodes to the nucleic acid labels on the affinity reagents. Techniques of library preparation, sequencing, and bioinformatics may then be used to group the sequences according to cell/discrete entity barcodes. Any suitable affinity reagent that can bind to or recognize a biological sample or portion or component thereof, such as a protein, a molecule, or complexes thereof, may be utilized in connection with these methods. The affinity reagents may be labeled with nucleic acid sequences that relates their identity, e.g., the target specificity of the antibodies, permitting their detection and quantitation using the barcoding and sequencing methods described herein. Exemplary affinity reagents can include, for example, antibodies, antibody fragments, Fabs, scFvs, peptides, drugs, etc. or combinations thereof. The affinity reagents, e.g., antibodies, can be expressed by one or more organisms or provided using a biological synthesis technique, such as phage, mRNA, or ribosome display. The affinity reagents may also be generated via chemical or biochemical means, such as by chemical linkage using N-Hydroxysuccinimide (NETS), click chemistry, or streptavidin-biotin interaction, for example. The oligo-affinity reagent conjugates can also be generated by attaching oligos to affinity reagents and hybridizing, ligating, and/or extending via polymerase, etc., additional oligos to the previously conjugated oligos. An advantage of affinity reagent labeling with nucleic acids is that it permits highly multiplexed analysis of biological samples. For example, large mixtures of antibodies or binding reagents recognizing a variety of targets in a sample can be mixed together, each labeled with its own nucleic acid sequence. This cocktail can then be reacted to the sample and subjected to a barcoding workflow as described herein to recover information about which reagents bound, their quantity, and how this varies among the different entities in the sample, such as among single cells. The above approach can be applied to a variety of molecular targets, including samples including one or more of cells, peptides, proteins, macromolecules, macromolecular complexes, etc. The sample can be subjected to conventional processing for analysis, such as fixation and permeabilization, aiding binding of the affinity reagents. To obtain highly accurate quantitation, the unique molecular identifier (UMI) techniques described herein can also be used so that affinity reagent molecules are counted accurately. This can be accomplished in a number of ways, including by synthesizing UMIs onto the labels attached to each affinity reagent before, during, or after conjugation, or by attaching the UMIs microfluidically when the reagents are used. Similar methods of generating the barcodes, for example, using combinatorial barcode techniques as applied to single cell sequencing and described herein, are applicable to the affinity reagent technique. These techniques enable the analysis of proteins and/or epitopes in a variety of biological samples to perform, for example, mapping of epitopes or post translational modifications in proteins and other entities or performing single cell proteomics. For example, using the methods described herein, it is possible to generate a library of labeled affinity reagents that detect an epitope in all proteins in the proteome of an organism, label those epitopes with the reagents, and apply the barcoding and sequencing techniques described herein to detect and accurately quantitate the labels associated with these epitopes.

A number of nucleic acid polymerases can be used in the amplification reactions utilized in certain embodiments provided herein, including any enzyme that can catalyze the polymerization of nucleotides (including analogs thereof) into a nucleic acid strand. Such nucleotide polymerization can occur in a template-dependent fashion. Such polymerases can include without limitation naturally occurring polymerases and any subunits and truncations thereof, mutant polymerases, variant polymerases, recombinant, fusion or otherwise engineered polymerases, chemically modified polymerases, synthetic molecules or assemblies, and any analogs, derivatives or fragments thereof that retain the ability to catalyze such polymerization. Optionally, the polymerase can be a mutant polymerase comprising one or more mutations involving the replacement of one or more amino acids with other amino acids, the insertion or deletion of one or more amino acids from the polymerase, or the linkage of parts of two or more polymerases. Typically, the polymerase comprises one or more active sites at which nucleotide binding and/or catalysis of nucleotide polymerization can occur. Some exemplary polymerases include without limitation DNA polymerases and RNA polymerases. The term “polymerase” and its variants, as used herein, also includes fusion proteins comprising at least two portions linked to each other, where the first portion comprises a peptide that can catalyze the polymerization of nucleotides into a nucleic acid strand and is linked to a second portion that comprises a second polypeptide. In some embodiments, the second polypeptide can include a reporter enzyme or a processivity-enhancing domain. Optionally, the polymerase can possess 5′ exonuclease activity or terminal transferase activity. In some embodiments, the polymerase can be optionally reactivated, for example through the use of heat, chemicals or re-addition of new amounts of polymerase into a reaction mixture. In some embodiments, the polymerase can include a hot-start polymerase or an aptamer-based polymerase that optionally can be reactivated.

In various embodiments, the nucleic acid amplification process generates amplicons that have incorporated within them a barcode nucleic acid identification sequence. In various embodiments, a ‘barcode’ nucleic acid identification sequence can be incorporated into a nucleic acid primer or linked to a primer to enable independent sequencing and identification to be associated with one another via a barcode which relates information and identification that originated from molecules that existed within the same sample. There are numerous techniques that can be used to attach barcodes to the nucleic acids within a discrete entity. For example, the target nucleic acids may or may not be first amplified and fragmented into shorter pieces. The molecules can be combined with discrete entities, e.g., droplets, containing the barcodes. The barcodes can then be attached to the molecules using, for example, splicing by overlap extension. In this approach, the initial target molecules can have “adaptor” sequences added, which are molecules of a known sequence to which primers can be synthesized. When combined with the barcodes, primers can be used that are complementary to the adaptor sequences and the barcode sequences, such that the product amplicons of both target nucleic acids and barcodes can anneal to one another and, via an extension reaction such as DNA polymerization, be extended onto one another, generating a double-stranded product including the target nucleic acids attached to the barcode sequence. Alternatively, the primers that amplify that target can themselves be barcoded so that, upon annealing and extending onto the target, the amplicon produced has the barcode sequence incorporated into it. This can be applied with a number of amplification strategies, including specific amplification with PCR or non-specific amplification with, for example, MDA. An alternative enzymatic reaction that can be used to attach barcodes to nucleic acids is ligation, including blunt or sticky end ligation. In this approach, the DNA barcodes are incubated with the nucleic acid targets and ligase enzyme, resulting in the ligation of the barcode to the targets. The ends of the nucleic acids can be modified as needed for ligation by a number of techniques, including by using adaptors introduced with ligase or fragments to enable greater control over the number of barcodes added to the end of the molecule.

A barcode sequence can additionally be incorporated into microfluidic beads to decorate the bead with identical sequence tags. Such tagged beads can be inserted into microfluidic droplets and via droplet PCR amplification, tag each target amplicon with the unique bead barcode. Such barcodes can be used to identify specific droplets upon a population of amplicons originated from. This scheme can be utilized when combining a microfluidic droplet containing single individual cell with another microfluidic droplet containing a tagged bead. Upon collection and combination of many microfluidic droplets, amplicon sequencing results allow for assignment of each product to unique microfluidic droplets. In a typical implementation, we use barcodes on the Mission Bio Tapestri™ beads to tag and then later identify each droplet's amplicon content. The use of barcodes is described in U.S. patent application Ser. No. 15/940,850 filed March 29, 2018 by Abate, A. et al., entitled ‘Sequencing of Nucleic Acids via Barcoding in Discrete Entities’, incorporated by reference herein.

In some embodiments, it may be advantageous to introduce barcodes into discrete entities, e.g., microdroplets, on the surface of a bead, such as a solid polymer bead or a hydrogel bead. These beads can be synthesized using a variety of techniques. For example, using a mix-split technique, beads with many copies of the same, random barcode sequence can be synthesized. This can be accomplished by, for example, creating a plurality of beads including sites on which DNA can be synthesized. The beads can be divided into four collections and each mixed with a buffer that will add a base to it, such as an A, T, G, or C. By dividing the population into four subpopulations, each subpopulation can have one of the bases added to its surface. This reaction can be accomplished in such a way that only a single base is added and no further bases are added. The beads from all four subpopulations can be combined and mixed together, and divided into four populations a second time. In this division step, the beads from the previous four populations may be mixed together randomly. They can then be added to the four different solutions, adding another, random base on the surface of each bead. This process can be repeated to generate sequences on the surface of the bead of a length approximately equal to the number of times that the population is split and mixed. If this was done 10 times, for example, the result would be a population of beads in which each bead has many copies of the same random 10-base sequence synthesized on its surface. The sequence on each bead would be determined by the particular sequence of reactors it ended up in through each mix-spit cycle.

A barcode may further comprise a ‘unique identification sequence’ (UMI). A UMI is a nucleic acid having a sequence which can be used to identify and/or distinguish one or more first molecules to which the UMI is conjugated from one or more second molecules. UMIs are typically short, e.g., about 5 to 20 bases in length, and may be conjugated to one or more target molecules of interest or amplification products thereof. UMIs may be single or double stranded. In some embodiments, both a nucleic acid barcode sequence and a UMI are incorporated into a nucleic acid target molecule or an amplification product thereof. Generally, a UMI is used to distinguish between molecules of a similar type within a population or group, whereas a nucleic acid barcode sequence is used to distinguish between populations or groups of molecules. In some embodiments, where both a UMI and a nucleic acid barcode sequence are utilized, the UMI is shorter in sequence length than the nucleic acid barcode sequence.

In some implementations, solid supports, beads, and the like are coated with affinity reagents. Affinity reagents include, without limitation, antigens, antibodies or aptamers with specific binding affinity for a target molecule. The affinity reagents bind to one or more targets within the single cell entities. Affinity reagents are often detectably labeled (e.g., with a fluorophore). Affinity reagents are sometimes labeled with unique barcodes, oligonucleotide sequences, or UMI's.

In one particular implementation, a solid support contains a plurality of affinity reagents, each specific for a different target molecule but containing a common sequence to be used to identify the unique solid support. Affinity reagents that bind a specific target molecule are collectively labeled with the same oligonucleotide sequence such that affinity molecules with different binding affinities for different targets are labeled with different oligonucleotide sequences. In this way, target molecules within a single target entity are differentially labeled in these implements to determine which target entity they are from but contain a common sequence to identify them from the same solid support.

### Example System and/or Computer Embodiments

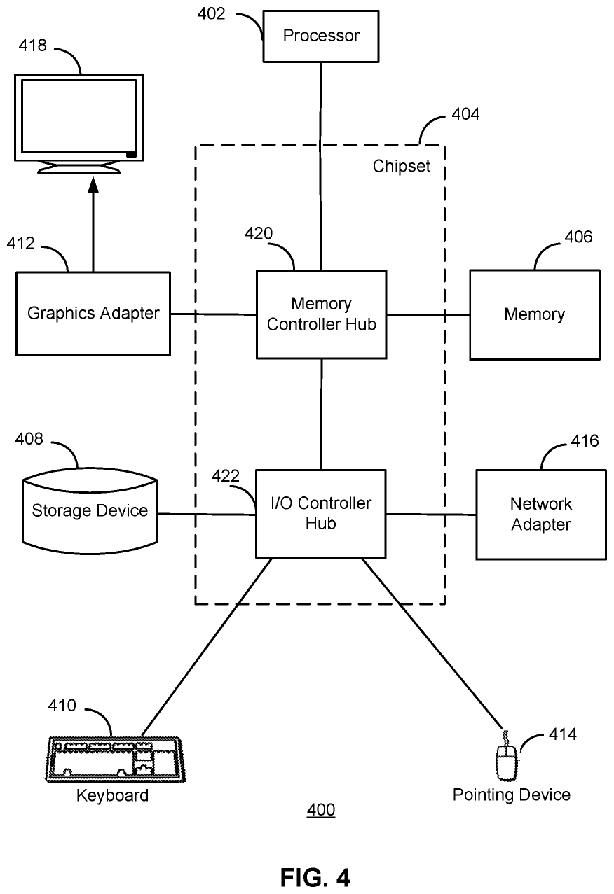

FIG. 4 depicts an example computing device 400 for implementing system and methods described in reference to FIGS. 1-3A/3B. For example, the example computing device 400 is configured to perform all or a portion of the steps shown in FIG. 2 corresponding to the amplicon design workflow. Examples of a computing device can include a personal computer, desktop computer laptop, server computer, a computing node within a cluster, message processors, hand-held devices, multi-processor systems, microprocessor-based or programmable consumer electronics, network PCs, minicomputers, mainframe computers, mobile telephones, PDAs, tablets, pagers, routers, switches, and the like.

In some embodiments, the computing device 400 includes at least one processor 402 coupled to a chipset 404. The chipset 404 includes a memory controller hub 420 and an input/output (I/O) controller hub 422. A memory 406 and a graphics adapter 412 are coupled to the memory controller hub 420, and a display 418 is coupled to the graphics adapter 412. A storage device 408, an input interface 414, and network adapter 416 are coupled to the I/O controller hub 422. Other embodiments of the computing device 400 have different architectures.

The storage device 408 is a non-transitory computer-readable storage medium such as a hard drive, compact disk read-only memory (CD-ROM), DVD, or a solid-state memory device. The memory 406 holds instructions and data used by the processor 402. The input interface 414 is a touch-screen interface, a mouse, track ball, or other type of input interface, a keyboard, or some combination thereof, and is used to input data into the computing device 400. In some embodiments, the computing device 400 may be configured to receive input (e.g., commands) from the input interface 414 via gestures from the user. The graphics adapter 412 displays images and other information on the display 418. For example, the display 418 can show metrics pertaining to the generated libraries (e.g., DNA or RNA libraries) and/or any characterization of single cells. The network adapter 416 couples the computing device 400 to one or more computer networks.

The computing device 400 is adapted to execute computer program modules for providing functionality described herein. As used herein, the term “module” refers to computer program logic used to provide the specified functionality. Thus, a module can be implemented in hardware, firmware, and/or software. In one embodiment, program modules are stored on the storage device 408, loaded into the memory 406, and executed by the processor 402.

The types of computing devices 400 can vary from the embodiments described herein. For example, the computing device 400 can lack some of the components described above, such as graphics adapters 412, input interface 414, and displays 418. In some embodiments, a computing device 400 can include a processor 402 for executing instructions stored on a memory 406.

The methods of aligning sequence reads and characterizing cells can be implemented in hardware or software, or a combination of both. In one embodiment, a non-transitory machine-readable storage medium, such as one described above, is provided, the medium comprising a data storage material encoded with machine readable data which, when using a machine programmed with instructions for using said data, is capable of displaying any of the datasets and execution and results disclosed herein. Such data can be used for a variety of purposes, such as patient monitoring, treatment considerations, and the like. Embodiments of the methods described above can be implemented in computer programs executing on programmable computers, comprising a processor, a data storage system (including volatile and non-volatile memory and/or storage elements), a graphics adapter, an input interface, a network adapter, at least one input device, and at least one output device. A display is coupled to the graphics adapter. Program code is applied to input data to perform the functions described above and generate output information. The output information is applied to one or more output devices, in known fashion. The computer can be, for example, a personal computer, microcomputer, or workstation of conventional design.

Each program can be implemented in a high level procedural or object oriented programming language to communicate with a computer system. However, the programs can be implemented in assembly or machine language, if desired. In any case, the language can be a compiled or interpreted language. Each such computer program is preferably stored on a storage media or device (e.g., ROM or magnetic diskette) readable by a general or special purpose programmable computer, for configuring and operating the computer when the storage media or device is read by the computer to perform the procedures described herein. The system can also be considered to be implemented as a computer-readable storage medium, configured with a computer program, where the storage medium so configured causes a computer to operate in a specific and predefined manner to perform the functions described herein.

The signature patterns and databases thereof can be provided in a variety of media to facilitate their use. “Media” refers to a manufacture that contains the signature pattern information of the present invention. The databases of the present invention can be recorded on computer readable media, e.g. any medium that can be read and accessed directly by a computer. Such media include, but are not limited to: magnetic storage media, such as floppy discs, hard disc storage medium, and magnetic tape; optical storage media such as CD-ROM; electrical storage media such as RAM and ROM; and hybrids of these categories such as magnetic/optical storage media. One of skill in the art can readily appreciate how any of the presently known computer readable mediums can be used to create a manufacture comprising a recording of the present database information. “Recorded” refers to a process for storing information on computer readable medium, using any such methods as known in the art. Any convenient data storage structure can be chosen, based on the means used to access the stored information. A variety of data processor programs and formats can be used for storage, e.g. word processing text file, database format, etc.

In various embodiments, the different algorithms of FIGS. 2, 3A, 3B, and 3C may be implemented with machine language (software) in a microprocessor environment (hardware). In an exemplary embodiment of the disclosure, machine learning models can be trained to identify data trends and relationships between attributes such that correlated attributes may be identified and separated from independent attributes. Similarly, the statistical analysis may be implemented in software, hardware or a combination of software and hardware. An exemplary implementation includes instruction which may be stored at one or more memory circuitries and executed on one or more processor circuitries to implement the principles disclosed herein. The following is a brief description of such exemplary systems for implementing the disclosed principles. It should be noted that the disclosed embodiments are exemplary and non-limiting.

An exemplary embodiment of the disclosure comprises the steps of (A) data preparation, and (B) the iterative training and testing of a machine learning model. The data preparation step comprises: (1) Providing training data table input set to form an input data set; the table comprising a plurality of amplicons with each amplicon having an identifier; (2) providing a plurality of attributes and a performance indicators for each amplicon; and (3) selecting a classification model (e.g., random forest) to select a key subset of attributes from among the plurality of attributes to generate a subset input data; (a table with 5-6 column and the performance column).

The iterative training and testing of the model comprises: (1) randomly splitting the subset input data set to two groups: (a) training dataset, and (b) testing dataset; (2) training the model on the training dataset to associate one or more feature of the subset of input data with the performance label to obtain a predictive factor; (3) evaluating accuracy of the predictive factor using testing dataset.

### EXAMPLES

### Example 1: Example Amplicon Design Process Improves DNA Panel Performance

In an exemplary implementation, 10 different DNA panels were designed with amplicons spanning a wide range of design properties. The tested amplicons are classified into low, average or high performer amplicons based on their normalized reads-per-cell value. The design properties of the amplicons are the features.

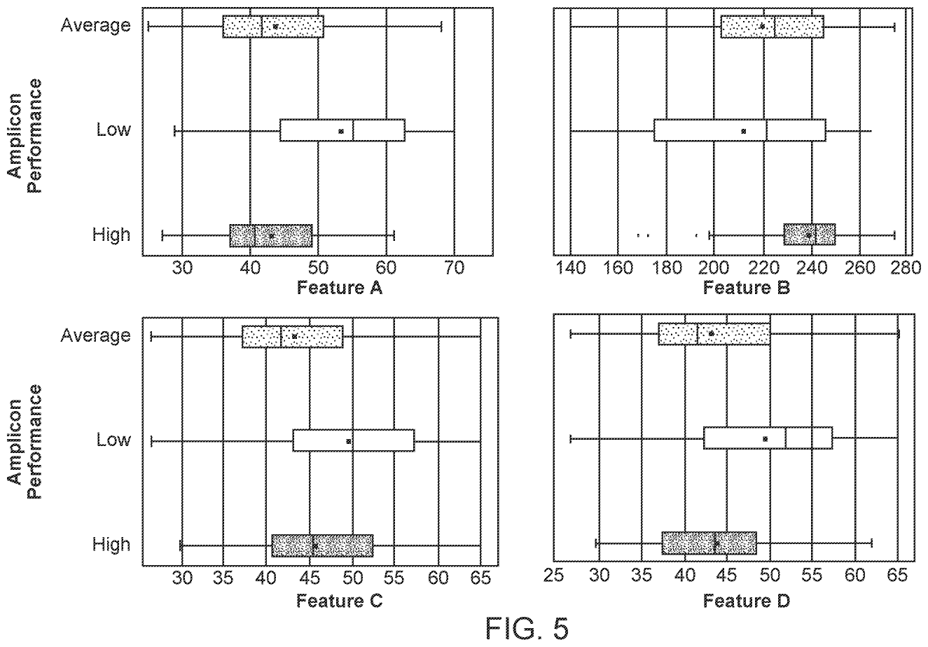

FIG. 5 depicts example box plots showing different categories (e.g., low, average, high) of amplicons based on values for four different amplicon features. For example, the box plots of FIG. 5 show that the “high” performing amplicons generally have a higher value for Feature B in comparison to the Feature B value for “average” and “low” performing amplicons. As another example, “low” performing amplicons generally have higher values for Feature A, Feature C, and Feature D in comparison to the corresponding Feature A, Feature C, and Feature D values for “average” and “high” performing amplicons.

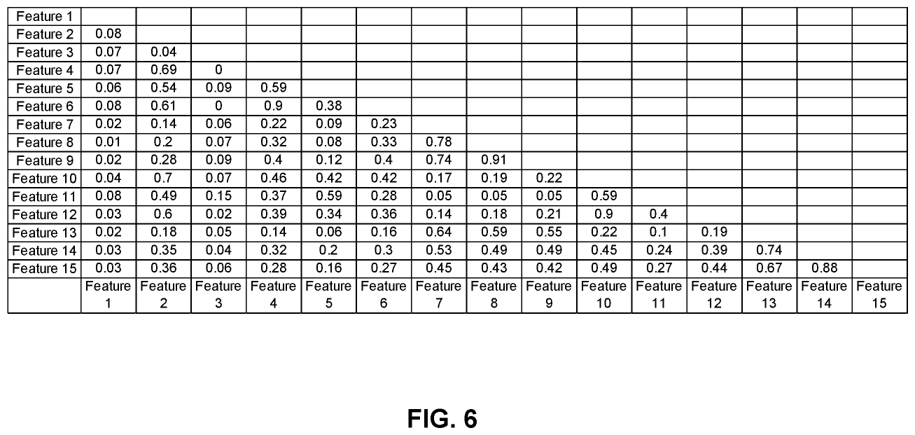

Highly correlated features were identified and pruned. For example, FIG. 6 depicts example correlations between different amplicon features. Only independent features were kept for feature distribution analysis and building prediction models. For example, if the correlation between two features was greater than 0.5, then only one of the two features was kept whereas the other feature was removed.

Top amplicon features (e.g., key attributes) were identified using two different feature selection methods. For example, the first method involved recursive feature elimination (RFE) whereas the second method involved selecting amplicon features that were most heavily weighted in a model (e.g., random forest classifier). Statistical values (e.g., mean and/or range) of the top amplicon features were analyzed and their significance of variance were determined between classes. These ranges of the top amplicon features were then used as parameters for designing new panels including improved amplicons underlying the Tapestri® Designer. For example, the improved amplicons were designed with amplicon features based on the statistical measures of the top amplicon features. As a specific example, the improved amplicons were designed with features that fell within the range of the top amplicon features. As another example, the improved amplicons were designed with a feature value that was the mean value of the top amplicon features.

To test the performance of new panels including the improved amplicons with the selected attributes, small (31), medium (128) and large (287) amplicon panels were constructed. Multiple runs were conducted for each panel with different cell types. Overall, the small, medium, and large amplicon panels exhibited high panel performance of 97%, 92% and 88% across the three panels. Additionally, using the new amplicons resulted in approximately 10-20% improvement in panel uniformity.

### Example 2: Prediction Models for Validating Designed Amplicons

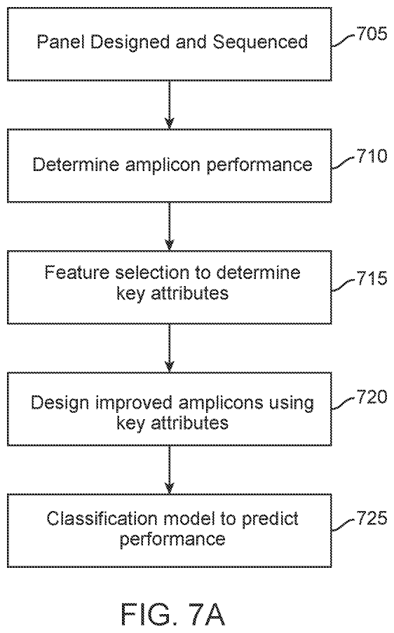

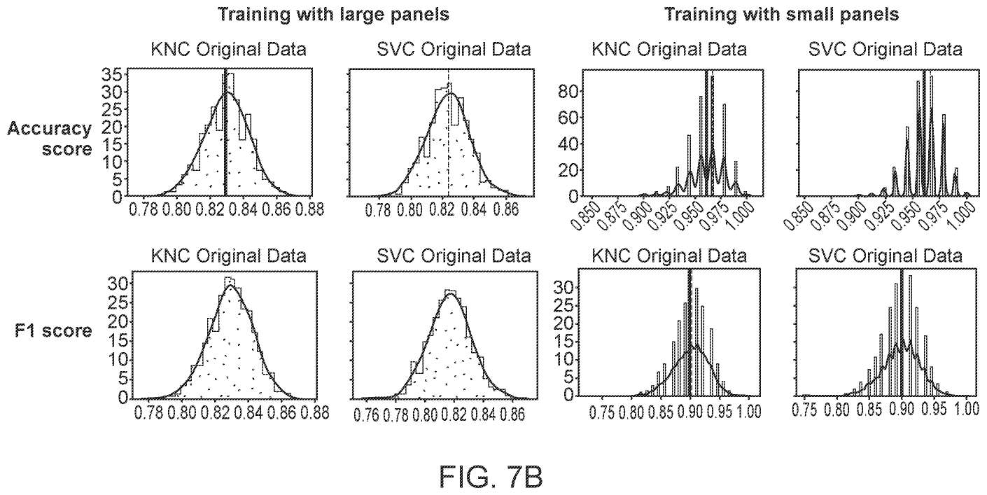

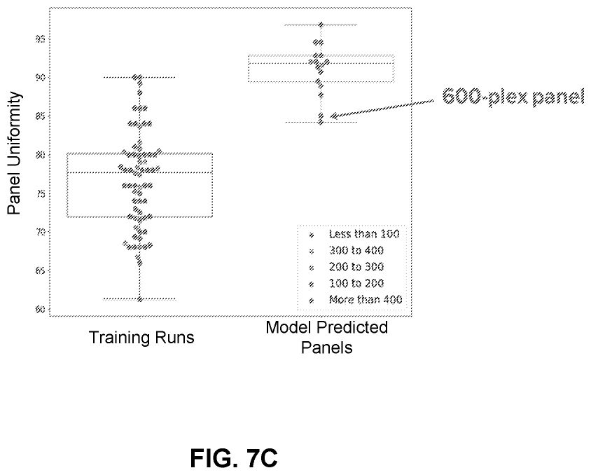

FIG. 7A shows an example process including feature selection of key attributes and in silico validation of amplicons designed based on the key attributes. Here, at step 705, the panel of amplicons was designed and amplicons were sequenced. At 710, the performance of the amplicons were determined. The performance of the amplicons included the extent of coverage, panel uniformity, and normalized read value for the amplicon. At step 715, a feature selection process was performed to identify key attributes of the amplicons. Here, the feature selection process involves two feature selection methods. The first method involved performing a recursive feature elimination (RFE) to identify features and the second method involved selecting amplicon features that were most heavily weighted in a model (e.g., random forest classifier). The key attributes of the amplicons represent the amplicon attributes that were identified by both feature selection methods. Highly influential attributes were identified, including example attributes such as amplicon-GC, amplicon-length, and primer-GC. Step 720 involves designing improved amplicons using the key attributes. Step 725 involves an in silico validation of the improved amplicons using a classification model to predict the performance of the improved amplicons. Upon validation, the improved amplicons were included in a sequencing panel.

FIG. 7B depicts performance data (e.g., accuracy and F1 score) of the prediction model that was trained on differing panels (e.g., small versus large panels). Two prediction models (K Neighbors Classifier (KNC) and Support vector classification (SVC) models) with K-fold cross validation were trained with 10000 splits of 70/30 for training/testing dataset split, while all splits keep the same ratio of classes in both training and testing datasets. Average accuracy ranges from 0.80-0.88 for large dataset to 0.90-0.98 for small panels.

FIG. 7C depicts example performance data (e.g., panel uniformity) of the prediction model across differently sized panels. “Training runs” refer to datasets corresponding to amplicons categorized with labels of low, average, high performance. Thus, the panel uniformity measurement refers to panels that have not undergone the amplicon design workflow. Here, the box plot depicts a median of ˜77% panel uniformity with minimum and maximum uniformity values of ˜61% and ˜90% panel uniformity.

In contrast, the amplicon designer workflow was implemented to develop new panels including improved amplicons. These panels were also evaluated according to their performance (e.g., panel uniformity). As shown in FIG. 7C, these panels exhibited significantly improved amplicon performance and uniformity in targeted assay design across different panel size and genomic contents (human and mouse genomes). Three newly designed panels were sequenced. Multiple runs were conducted for each panel.

Generally, the larger panels (e.g., panels with more than 400 amplicons) were predicted by the classification model to exhibit lower panel uniformity than smaller panels (e.g., panels with less than 100 amplicons). Overall, the panels developed using the amplicon designer workflow achieved a median of ˜92% panel uniformity with minimum and maximum uniformity values of ˜84% and 97%.

### Example 3: DNA Panel with RNA Fusion Amplicons

RNA fusion amplicons were designed for 3 BCR-ABL1 fusion transcripts according to the workflow described in FIG. 2. The improved RNA fusion amplicons were included in a RNA panel and used to analyze known cell lines (e.g., K562, TOM-1, KCL-22, and KG1).

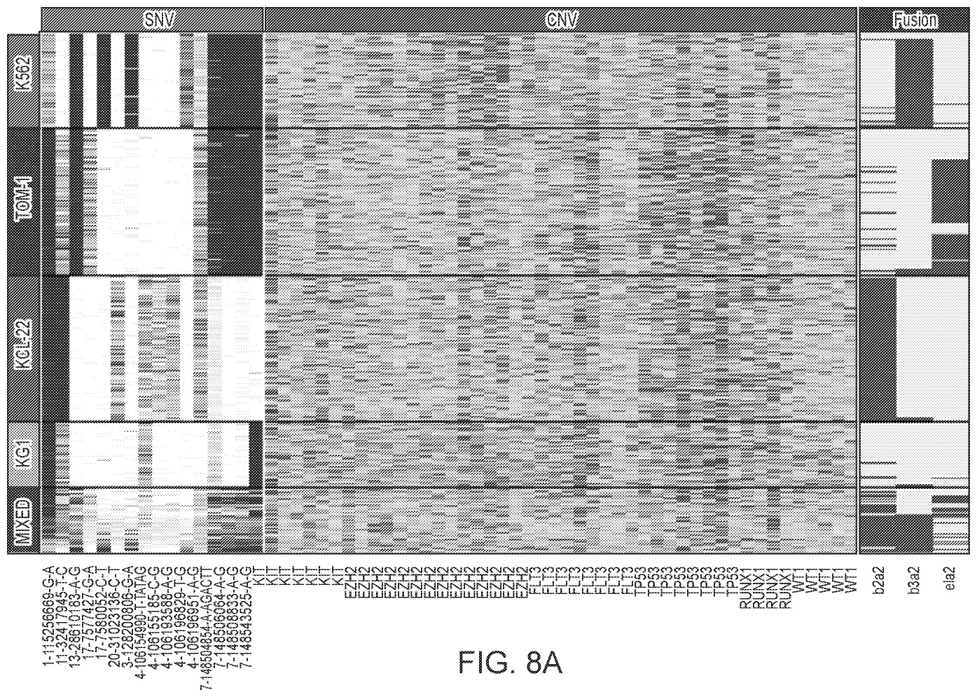

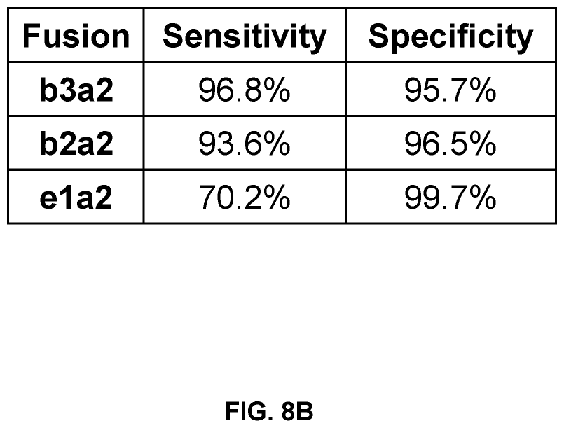

A 4 cell line mixture was run on the Tapestri platform with an acute myeloid leukemia (AML) DNA panel and primers to detect 3 BCR-ABL1 fusion transcripts. The data was resolved into 3 modalities of SNVs, CNVs and Fusions. K562 is positive for b3a2, TOM-1 is positive for e1a2 fusion, KCL-22 is positive for b2a2 fusion and KG1 was negative for all 3 fusions. The cells in the cell mixture were distinguished according to the SNV and CNV data, and the fusion data further correlated with the clustering. Specifically, FIG. 8A depicts a heat map for a DNA panel with RNA fusion amplicons that were designed using the amplicon design workflow. As expected, the RNA fusion amplicons in the panel were able to detect presence of b3a2 RNA fusions in K562 cells, presence of b2a2 RNA fusions in KCL-22 cells, presence of e1a2 RNA fusions in TOM-1 cells, and no RNA fusions in KG1 cells. A mixed cell population was observed which shows average of other cell lines in SNV, CNV and fusions.

FIG. 8B depicts performance (e.g., sensitivity and specificity) metrics for detecting three different RNA fusions using the amplicon design workflow. Here, a threshold of 20 reads per cell per fusion transcript was used to define a positive call. The sensitivity and specificity per fusion transcript across all cells was calculated. Notably, very high specificity was observed for all the RNA fusions (>95.7%). Furthermore, high sensitivity was observed for b3a2 and b2a2 (>93.6%) RNA fusions and good sensitivity was observed for e1a2 (70.2%) RNA fusions.

Altogether, the machine learning model generated panels exhibit more uniform amplification across amplicons. Furthermore, the amplicon design workflow (e.g., workflow shown in FIG. 2) was used to design amplicons for multiple genomes (human and mouse) and also of varying panel sizes. The RNA fusion amplicons designed using the amplicon design workflow exhibit high sensitivity, specificity, and align with SNV/CNV data of known cell lines.

The references made to the Tapestri® instrument are illustrative and non-limiting. The disclosed principles may be implemented with other instruments and/or systems without departing from the disclosed principles. It is further noted that the disclosed examples are merely illustrative and non-limiting of the principles. Other applicants of the disclosed principles can be made without departing from the spirit of the disclosed principles.

What is claimed is:

1. A method for designing a panel of RNA fusion amplicons, the method comprising:
providing a plurality of RNA fusion amplicons having a plurality of initial attributes, the RNA fusion amplicons representing one or more RNA fusions;
sequencing the plurality of RNA fusion amplicons with a targeted RNA panel;
selecting a subset of the plurality of RNA fusion amplicons according to performance of the subset of RNA fusion amplicons;
performing a feature selection among the subset of RNA fusion amplicons to select key attributes from the plurality of initial attributes, and
designing a plurality of improved RNA fusion amplicons comprising candidate attributes that are selected based on the key attributes of the subset of RNA fusion amplicons; and
validating the plurality of improved RNA fusion amplicons.

2. The method of claim 1, wherein performing a feature selection among the subset of RNA fusion amplicons to select key attributes from the plurality of initial attributes further comprises applying a ranking model.

3. The method of claim 2, wherein the ranking model implements a Recursive Feature Elimination (RFE) technique.

4. The method of claim 2, wherein performing a feature selection among the subset of RNA fusion amplicons to select key attributes from the plurality of initial attributes further comprises applying a second model.

5. The method of claim 4, wherein the second model comprises a weighted model.

6. The method of claim 5, wherein the selected key attributes represent attributes that are selected by both the ranking model and the second model.

7. The method of any one of claims 1-6, wherein performing the feature selection further comprises:
selecting key attributes representing independent attributes from highest importance attributes.

8. The method of claim 1, further comprising calculating a plurality of statistical parameters from the key attributes.

9. The method of claim 8, wherein designing the plurality of improved RNA fusion amplicons comprising attributes that are selected based on the key attributes comprises designing the plurality of improved RNA fusion amplicons to include one or more of the plurality of statistical parameters calculated from the key attributes.

10. The method of any one of claims 1-9, wherein validating the plurality of improved RNA fusion amplicons comprises sequencing the plurality of improved RNA fusion amplicons and determining a performance of the improved RNA fusion amplicons.

11. The method of any one of claims 1-9, wherein validating the plurality of improved RNA fusion amplicons comprises applying a predictive model to the plurality of improved RNA fusion amplicons, the predictive model trained to predict a performance of RNA fusion amplicons.

12. The method of claim 10 or 11, wherein the performance is a measure of panel uniformity.

13. The method of claim 10 or 11, wherein the performance is a sensitivity or specificity of detection of a presence or absence of a RNA fusion using the plurality of improved RNA fusion amplicons.

14. The method of any one of claims 1-13, wherein providing the plurality of RNA fusion amplicons having a plurality of initial attributes comprises constructing at least one fusion sequence.

15. The method of claim 14, wherein constructing the at least one fusion sequence comprises:
obtaining a sequence of a first gene and a sequence of a second gene;
identifying a fusion breakpoint in the sequence for the first gene and a fusion breakpoint in the sequence for the second gene;
concatenating the sequence of the first gene at the fusion breakpoint for the first gene with the sequence of the second gene at the fusion breakpoint for the second gene;
stitching together exon sequences of the first gene and the exon sequences of the second gene that flank the concatenated sequences at the fusion breakpoints.

16. A method for designing a panel of amplicons, the method comprising:
providing a plurality of amplicons having a plurality of initial attributes;
sequencing the plurality of amplicons with a single cell panel;
selecting a subset of the plurality of amplicons according to performance of the subset of amplicons;
performing a feature selection among the subset of amplicons to select key attributes from the plurality of initial attributes, and
designing a plurality of improved amplicons wherein the improved amplicons comprise attributes designed based on the selected key attributes of the subset of amplicons; and
validating the plurality of secondary amplicons.

17. The method of claim 16, wherein performing a feature selection among the subset of amplicons to select key attributes from the plurality of initial attributes further comprises applying a ranking model.

18. The method of claim 17, wherein the ranking model implements a Recursive Feature Elimination (RFE) technique.

19. The method of claim 17, wherein performing a feature selection among the subset of amplicons to select key attributes from the plurality of initial attributes further comprises applying a second model.

20. The method of claim 19, wherein the second model comprises a weighted model.

21. The method of claim 20, wherein the selected key attributes represent attributes that are selected by both the ranking model and the second model.

22. The method of any one of claims 16-21, wherein performing the feature selection further comprises:
selecting key attributes representing independent attributes from highest importance attributes.

23. The method of claim 16, further comprising calculating a plurality of statistical parameters from the key attributes.

24. The method of claim 23, wherein designing the plurality of improved amplicons comprising attributes that are selected based on the key attributes comprises designing the plurality of improved amplicons to include one or more of the plurality of statistical parameters calculated from the key attributes.

25. The method of any one of claims 16-24, wherein validating the plurality of improved amplicons comprises sequencing the plurality of improved amplicons and determining a performance of the improved amplicons.

26. The method of any one of claims 16-24, wherein validating the plurality of improved amplicons comprises applying a predictive model to the plurality of improved amplicons, the predictive model trained to predict a performance of amplicons.

27. The method of claim 25 or 26, wherein the performance is a measure of panel uniformity.

28. The method of claim 25 or 26, wherein the performance is a sensitivity or specificity of detection of a presence or absence of a RNA fusion using the plurality of improved RNA fusion amplicons.

29. The method of any one of claims 25-28, wherein responsive to the validation determining that the plurality of improved amplicons fails to meet a pre-determined performance metric, re-analyzing the improved amplicons using an amplicon design workflow to generate further improved amplicons.

30. The method of any one of claims 16-29, wherein the single cell panel is a targeted RNA panel, a targeted DNA panel, a whole genome panel, or whole transcriptome panel.

31. The method of any one of claims 16-30, wherein the plurality of amplicons and the plurality of improved amplicons are DNA amplicons.

32. The method of any one of claims 16-30, wherein the plurality of amplicons and the plurality of improved amplicons are RNA fusion amplicons.

33. The method of claim 32, wherein providing a plurality of amplicons having a plurality of initial attributes further comprises constructing at least one fusion sequence.

34. The method of claim 33, wherein constructing the at least one fusion sequence comprises:
obtaining a sequence of a first gene and a sequence of a second gene;
identifying a fusion breakpoint in the sequence for the first gene and a fusion breakpoint in the sequence for the second gene;
concatenating the sequence of the first gene at the fusion breakpoint for the first gene with the sequence of the second gene at the fusion breakpoint for the second gene;
stitching together exon sequences of the first gene and the exon sequences of the second gene that flank the concatenated sequences at the fusion breakpoints.

35. The method of any one of claim 1-14 or 32, wherein the improved RNA fusion amplicons are designed according to a BCR-ABL RNA fusion.

36. The method of claim 35, wherein the BCR-ABL RNA fusion is any one of a b3a2 RNA fusion, b2a2 RNA fusion, or e1a2 RNA fusion.

37. The method of claim 36, wherein the BCR-ABL RNA fusion is a b3a2 RNA fusion, and wherein the improved RNA fusion amplicons achieve at least a 90% sensitivity.

38. The method of claim 36, wherein the BCR-ABL RNA fusion is a b3a2 RNA fusion, and wherein the improved RNA fusion amplicons achieve at least a 90% specificity.

39. The method of claim 36, wherein the BCR-ABL RNA fusion is a b2a2 RNA fusion, and wherein the improved RNA fusion amplicons achieve at least a 90% sensitivity.

40. The method of claim 36, wherein the BCR-ABL RNA fusion is a b2a2 RNA fusion, and wherein the improved RNA fusion amplicons achieve at least a 90% specificity.

41. The method of claim 36, wherein the BCR-ABL RNA fusion is a e1a2 RNA fusion, and wherein the improved RNA fusion amplicons achieve at least a 70% sensitivity.

42. The method of claim 36, wherein the BCR-ABL RNA fusion is a e1a2 RNA fusion, and wherein the improved RNA fusion amplicons achieve at least a 90% specificity.

43. The method of any one of claims 1-42, wherein the initial attributes, key attributes, or candidate attributes of amplicons comprise characteristics of primers that are designed to target the amplicons.

44. The method of claim 43, wherein the initial attributes, key attributes, or candidate attributes are selected from a group consisting of a primer length, a percentage of GC content in a primer, a GC content at 3′ end of primer, a GC content at 5′ end of primer and a number of G or C bases within the last five bases of 3′ end of the primer.

45. A non-transitory computer readable medium for designing a panel of RNA fusion amplicons, the non-transitory computer readable medium comprising instructions that, when executed by a processor, cause the processor to:
provide a plurality of RNA fusion amplicons having a plurality of initial attributes, the RNA fusion amplicons representing one or more RNA fusions;
sequence the plurality of RNA fusion amplicons with a targeted RNA panel;
select a subset of the plurality of RNA fusion amplicons according to performance of the subset of RNA fusion amplicons;
perform a feature selection among the subset of RNA fusion amplicons to select key attributes from the plurality of initial attributes, and
design a plurality of improved RNA fusion amplicons comprising candidate attributes that are selected based on the key attributes of the subset of RNA fusion amplicons; and
validate the plurality of improved RNA fusion amplicons.

46. The non-transitory computer readable medium of claim 45, wherein the instructions that, when executed by a processor, cause the processor to perform a feature selection among the subset of RNA fusion amplicons to select key attributes from the plurality of initial attributes further comprises instructions that, when executed by the processor, cause the processor to apply a ranking model.

47. The non-transitory computer readable medium of claim 46, wherein the ranking model implements a Recursive Feature Elimination (RFE) technique.

48. The non-transitory computer readable medium of claim 47, wherein the instructions that, when executed by a processor, cause the processor to perform a feature selection among the subset of RNA fusion amplicons to select key attributes from the plurality of initial attributes further comprises instructions that, when executed by the processor, cause the processor to apply a second model.

49. The non-transitory computer readable medium of claim 48, wherein the second model comprises a weighted model.

50. The non-transitory computer readable medium of claim 49, wherein the selected key attributes represent attributes that are selected by both the ranking model and the second model.

51. The non-transitory computer readable medium of any one of claims 45-50, wherein the instructions that, when executed by a processor, cause the processor to perform the feature selection further comprises instructions that, when executed by the processor, cause the processor to:
select key attributes representing independent attributes from highest importance attributes.

52. The non-transitory computer readable medium of claim 45, wherein the instructions further comprise instructions that, when executed by the processor, cause the processor to calculate a plurality of statistical parameters from the key attributes.

53. The non-transitory computer readable medium of claim 52, wherein the instructions that, when executed by a processor, cause the processor to design the plurality of improved RNA fusion amplicons comprising attributes that are selected based on the key attributes further comprises instructions that, when executed by the processor, cause the processor to design the plurality of improved RNA fusion amplicons to include one or more of the plurality of statistical parameters calculated from the key attributes.

54. The non-transitory computer readable medium of any one of claims 45-53, wherein the instructions that, when executed by a processor, cause the processor to validate the plurality of improved RNA fusion amplicons further comprises instructions that, when executed by the processor, cause the processor to sequence the plurality of improved RNA fusion amplicons and determine a performance of the improved RNA fusion amplicons.

55. The non-transitory computer readable medium of any one of claims 45-53, wherein the instructions that, when executed by a processor, cause the processor to validate the plurality of improved RNA fusion amplicons further comprises instructions that, when executed by the processor, cause the processor to apply a predictive model to the plurality of improved RNA fusion amplicons, the predictive model trained to predict a performance of RNA fusion amplicons.

56. The non-transitory computer readable medium of claim 54 or 55, wherein the performance is a measure of panel uniformity.

57. The non-transitory computer readable medium of claim 54 or 55, wherein the performance is a sensitivity or specificity of detection of a presence or absence of a RNA fusion using the plurality of improved RNA fusion amplicons.

58. The non-transitory computer readable medium of any one of claims 45-57, wherein the instructions that cause the processor to provide the plurality of RNA fusion amplicons having a plurality of initial attributes further comprises instructions that, when executed by the processor, cause the processor to construct at least one fusion sequence.

59. The non-transitory computer readable medium of claim 58, wherein the instructions that, when executed by a processor, cause the processor to construct the at least one fusion sequence further comprises instructions that, when executed by the processor, cause the processor to:
obtain a sequence of a first gene and a sequence of a second gene;
identify a fusion breakpoint in the sequence for the first gene and a fusion breakpoint in the sequence for the second gene;
concatenate the sequence of the first gene at the fusion breakpoint for the first gene with the sequence of the second gene at the fusion breakpoint for the second gene;
stitch together exon sequences of the first gene and the exon sequences of the second gene that flank the concatenated sequences at the fusion breakpoints.

60. A non-transitory computer readable medium for designing a panel of amplicons comprising instructions that, when executed by a processor, cause the processor to:
provide a plurality of amplicons having a plurality of initial attributes;
sequence the plurality of amplicons with a single cell panel;
select a subset of the plurality of amplicons according to performance of the subset of amplicons;
perform a feature selection among the subset of amplicons to select key attributes from the plurality of initial attributes, and
design a plurality of improved amplicons wherein the improved amplicons comprise attributes designed based on the selected key attributes of the subset of amplicons; and
validate the plurality of secondary amplicons.

61. The non-transitory computer readable medium of claim 60, wherein the instructions that cause the processor to perform a feature selection among the subset of amplicons to select key attributes from the plurality of initial attributes further comprises instructions that, when executed by the processor, cause the processor to apply a ranking model.

62. The non-transitory computer readable medium of claim 61, wherein the ranking model implements a Recursive Feature Elimination (RFE) technique.

63. The non-transitory computer readable medium of claim 61 or 62, wherein the instructions that cause the processor to perform a feature selection among the subset of amplicons to select key attributes from the plurality of initial attributes further comprises instructions that, when executed by the processor, cause the processor to apply a second model.

64. The non-transitory computer readable medium of claim 63, wherein the second model comprises a weighted model.

65. The non-transitory computer readable medium of claim 63 or 64, wherein the selected key attributes represent attributes that are selected by both the ranking model and the second model.

66. The non-transitory computer readable medium of any one of claims 60-65, wherein the instructions that cause the processor to perform the feature selection further comprises instructions that, when executed by the processor, cause the processor to:
select key attributes representing independent attributes from highest importance attributes.

67. The non-transitory computer readable medium of claim 66, wherein the instructions further comprise instructions that, when executed by a processor, cause the processor to calculate a plurality of statistical parameters from the key attributes.

68. The non-transitory computer readable medium of claim 67, wherein the instructions that cause the processor to design the plurality of improved amplicons comprising attributes that are selected based on the key attributes further comprises instructions that, when executed by the processor, cause the processor to design the plurality of improved amplicons to include one or more of the plurality of statistical parameters calculated from the key attributes.

69. The non-transitory computer readable medium of any one of claims 60-68, wherein the instructions that cause the processor to validate the plurality of improved amplicons further comprises instructions that, when executed by the processor, cause the processor to sequence the plurality of improved amplicons and determine a performance of the improved amplicons.

70. The non-transitory computer readable medium of any one of claims 60-68, wherein instructions that cause the processor to validate the plurality of improved amplicons further comprises instructions that, when executed by the processor, cause the processor to apply a predictive model to the plurality of improved amplicons, the predictive model trained to predict a performance of amplicons.

71. The non-transitory computer readable medium of claim 69 or 70, wherein the performance is a measure of panel uniformity.

72. The non-transitory computer readable medium of claim 69 or 70, wherein the performance is a sensitivity or specificity of detection of a presence or absence of a RNA fusion using the plurality of improved RNA fusion amplicons.

73. The non-transitory computer readable medium of any one of claims 69-72, wherein responsive to the validation determining that the plurality of improved amplicons fails to meet a pre-determined performance metric, the instructions, when executed by the processor, cause the processor to re-analyze the improved amplicons using an amplicon design workflow to generate further improved amplicons.

74. The non-transitory computer readable medium of any one of claims 60-73, wherein the single cell panel is a targeted RNA panel, a targeted DNA panel, a whole genome panel, or whole transcriptome panel.

75. The non-transitory computer readable medium of any one of claims 60-74, wherein the plurality of amplicons and the plurality of improved amplicons are DNA amplicons.

76. The non-transitory computer readable medium of any one of claims 60-74, wherein the plurality of amplicons and the plurality of improved amplicons are RNA fusion amplicons.

77. The non-transitory computer readable medium of claim 76, wherein the instructions that cause the processor to provide a plurality of amplicons having a plurality of initial attributes further comprises instructions that when executed by the processor, cause the processor to construct at least one fusion sequence.

78. The non-transitory computer readable medium of claim 77, wherein the instructions that cause the processor to construct the at least one fusion sequence further comprises instructions that when executed by the processor, cause the processor to:
obtain a sequence of a first gene and a sequence of a second gene;
identify a fusion breakpoint in the sequence for the first gene and a fusion breakpoint in the sequence for the second gene;
concatenate the sequence of the first gene at the fusion breakpoint for the first gene with the sequence of the second gene at the fusion breakpoint for the second gene;
stitch together exon sequences of the first gene and the exon sequences of the second gene that flank the concatenated sequences at the fusion breakpoints.

79. The non-transitory computer readable medium of any one of claim 45-59 or 76, wherein the improved RNA fusion amplicons are designed according to a BCR-ABL RNA fusion.

80. The non-transitory computer readable medium of claim 79, wherein the BCR-ABL RNA fusion is any one of a b3a2 RNA fusion, b2a2 RNA fusion, or e1a2 RNA fusion.

81. The non-transitory computer readable medium of claim 80, wherein the BCR-ABL RNA fusion is a b3a2 RNA fusion, and wherein the improved RNA fusion amplicons achieve at least a 90% sensitivity.

82. The non-transitory computer readable medium of claim 80, wherein the BCR-ABL RNA fusion is a b3a2 RNA fusion, and wherein the improved RNA fusion amplicons achieve at least a 90% specificity.

83. The non-transitory computer readable medium of claim 80, wherein the BCR-ABL RNA fusion is a b2a2 RNA fusion, and wherein the improved RNA fusion amplicons achieve at least a 90% sensitivity.

84. The non-transitory computer readable medium of claim 80, wherein the BCR-ABL RNA fusion is a b2a2 RNA fusion, and wherein the improved RNA fusion amplicons achieve at least a 90% specificity.

85. The non-transitory computer readable medium of claim 80, wherein the BCR-ABL RNA fusion is a e1a2 RNA fusion, and wherein the improved RNA fusion amplicons achieve at least a 70% sensitivity.

86. The non-transitory computer readable medium of claim 80, wherein the BCR-ABL RNA fusion is a e1a2 RNA fusion, and wherein the improved RNA fusion amplicons achieve at least a 90% specificity.

87. The non-transitory computer readable medium of any one of claims 45-86, wherein the initial attributes, key attributes, or candidate attributes of amplicons comprise characteristics of primers that are designed to target the amplicons.

88. The non-transitory computer readable medium of claim 87, wherein the initial attributes, key attributes, or candidate attributes are selected from a group consisting of a primer length, a percentage of GC content in a primer, a GC content at 3′ end of primer, a GC content at 5′ end of primer and a number of G or C bases within the last five bases of 3′ end of the primer.
